(19) United States
(12) Patent Application Publication
(10) Pub. No.: US 2025/0108029 A1
(43) Pub. Date: Apr. 3, 2025

(54) INHIBITORS OF THE INTERACTION BETWEEN THE ROCK AND PDK1 PROTEIN KINASES

(71) Applicants: Université Paris Cite, Paris (FR); Centre National de la Recherche Scientifique, Paris (FR); Institut National de la Sante et de la Recherche Medicale, Paris (FR); Sorbonne Universite, Paris (FR)

(72) Inventors: Benoit SCHNEIDER, Antony (FR); Jean-Philippe HERBEUVAL, Paris (FR); Nassima BEKADDOUR BENATIA, Marmande (FR); Anne BAUDRY, Antony (FR); Mathéa PIETRI, Antony (FR); Nicolas PIETRANCOSTA, Montigny le Bretonneux (FR); Laurent LE CORRE, Bourg la Reine (FR); Rodolphe ALVES DE SOUSA, Ivry sur Seine (FR); Patricia BUSCA, L'Haÿ-les-Roses (FR); Christine GRAVIER-PELLETIER, Mennecy (FR)

(21) Appl. No.: 18/696,182

(22) PCT Filed: Sep. 26, 2022
(86) PCT No.: PCT/EP2022/076610

§ 371 Date: Mar. 27, 2024

(30) Foreign Application Priority Data
Sep. 27, 2021 (FR) FR2110136

(51) Int. Cl. A61K 31/277; A61K 31/415; A61K 31/437; A61K 45/06; A61P 29/00

(52) U.S. Cl. CPC A61K 31/277; A61K 31/415; A61K 31/437; A61K 45/06; A61P 29/00; C07C 2601/16

(57) ABSTRACT

The present invention relates to an inhibitor of the interaction between the ROCK and PDK1 protein kinases. The present invention also relates to a pharmaceutical composition comprising at least one inhibitor of the interaction between the ROCK and PDK1 protein kinases as an active ingredient and at least one pharmaceutically acceptable excipient and/or support and/or a diluent and/or a pharmaceutically acceptable carrier. The invention also relates to the use of said pharmaceutical composition in the prevention and/or treatment of inflammatory diseases, viral and/or bacterial infections and autoimmune diseases.

### TECHNICAL FIELD

The present invention relates to inhibitors of the interaction between the ROCK and PDK1 protein kinases and to the uses thereof as anti-inflammatory and anti-interferon agents. The present invention also relates to a pharmaceutical composition comprising an inhibitor of the interaction between the ROCK and PDK1 protein kinases and to the use thereof in the prevention and/or treatment of inflammatory diseases, viral and/or bacterial infections and autoimmune diseases.

### PRIOR ART

Inflammation is a physiological process that is beneficial for the body to the extent that it is transient. It makes it possible, for example, to combat bacterial or viral infections. Nevertheless, numerous diseases affecting several organs such as the intestine, the central nervous system, the osteoarticular apparatus, the skin, the lungs, etc. are associated with chronic tissue inflammation. Chronic inflammation alters tissue homeostasis, leading to functional anomalies such as digestive disorders, the amplification of neurological or neurodegenerative diseases, a motor handicap, skin lesions, respiratory failure, etc. Chronic tissue inflammation results from the continuous production of inflammatory cytokines (TNFα, interleukins, interferons) by the immune cells or related cells (microglia cells in the central nervous system) which leads to the deterioration of cells that are sensitive to inflammatory factors. Very few drugs (steroidal and non-steroidal) currently make it possible to contain the inflammatory response (aspirin, ibuprofen, paracetamol, corticoids), some of which generate significant side effects in humans.

Therefore, it appears necessary to have novel compounds capable of containing the inflammatory response, especially by preventing or blocking the synthesis of inflammatory cytokines, directly in the immune cells, in order to prevent and/or treat inflammatory diseases, viral and/or bacterial infections and autoimmune diseases, while reducing the risks of side effects, when these are administered to the patient.

### SUMMARY OF THE INVENTION

The present invention relates to a compound of formula (I):

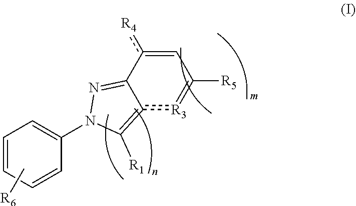

or a pharmaceutically acceptable salt thereof, for use in the prevention and/or treatment of a disease requiring the use of an inhibitor of the interaction between the ROCK protein kinase and the PDK1 protein kinase, wherein:
m is selected from 0, 1 and 2,
n is selected from 0, 1 and 2,
R₁ is a —COR₂ group, wherein R₂ is selected from the group consisting of an —OH group, an —NH₂ group, an alkoxy group, an alkylamine group, an arylalkylamine group, an —NH—NH₂ group, and a —CH₂—Ar group optionally substituted by at least one halogen atom, wherein Ar is an aryl group,
R₃ is a nitrogen atom, an —NHCOR₇ group, an —NHCOOR₈ group, or a —COSR₉ group, wherein R₇, R₈ and R₉ are C₁ to C₆ alkyl groups optionally substituted by at least one halogen atom,
R₄ is a nitrogen atom or an —NH₂ group,
R₅ and R₆, which are identical or different, are selected independently from each other in the group consisting of a hydrogen, an —OH group, an —NH group, an —NH₂ group, an alkoxy group, a thiol group, a sulfonamide group, an alkylamine group, and an aryl group optionally substituted by at least one halogen, and wherein:
the lines [symbol] and [symbol] , which are identical or different, depict, independently of each other, a single covalent bond or a triple covalent bond.

Quite surprisingly, the inventors have shown that the compounds of formula (I) are capable of binding to the interaction zone between the ROCK and PDK1 protein kinases, in particular on a specific protein domain of the ROCK protein kinase, advantageously on a specific protein domain of the ROCK1 protein kinase. By binding to this specific interaction zone, the compounds of formula (I) according to the present invention are capable of preventing and/or inhibiting and/or blocking the production and/or secretion by the immune cells of inflammatory cytokines, such as tumor necrosis factor alpha (TNFα), interleukins IL-1β, IL-6, IL-10, IL-12 p70, IL-18, IL-23, IL-33, but also the different subtypes of interferons (IFN), such as interferon alpha-2 (IFNα2), interferon gamma (IFNγ) and the MCP1 protein, without producing side effects.

The specific interaction zone between the ROCK and PDK1 protein kinases targeted by the compounds of formula (I) according to the present invention relates to the following amino acids of the ROCK1 protein kinase: proline 21 to glutamate 24 (P21-E24), tryptophan 122 to valine 136 (W122-V136), alanine 188 to phenylalanine 194 (A188-F194), lysine 222 (K222) and arginine 404 to tyrosine 405 (R404-Y405), the positions of the amino acids being established with respect to the amino acid sequences of the ROCK1 protein kinase (SEQ ID No. 11).

The following amino acids: lysine 222 (K222), phenylalanine 130 (F130), asparagine 132 (N132), serine 133 (S133), methionine 192 (M192) and arginine 404 (R404) are essential for the interaction with the compounds of formula (I) according to the present invention in biological terms, the positions of the amino acids being established with respect to the amino acid sequences of the ROCK1 protein kinase (SEQ ID No. 11).

Amino acid sequence of the ROCK1 protein kinase:
(SEQ ID No. 11)
GSLHMSFETRFEKMDNLLRDPKSEVNSDCLLDGLDALVYDLDFPALRKNK
NIDNFLSRYKDTINKIRDLRMKAEDYEVVKVIGRGAFGEVQLVRHKSTRK
VYAMKLLSKFEMIKRSDSAFFWEERDIMAFANSPWVVQLFYAFQDDRYLY
MVMEYMPGGDLVNLMSNYDVPEKWARFYTAEVVLALDAIHSMGFIHRDVK
PDNMLLDKSGHLKLADFGTCMKMNKEGMVRCDTAVGTPDYISPEVLKSQG
GDGYYGRECDWWSVGVFLYEMLVGDTPFYADSLVGTYSKIMNHKNSLTFP
DDNDISKEAKNLICAFLTDREVRLGRNGVEEIKRHLFFKNDQWAWETLRD
TVAPWPDLSSDIDTSNFDDLEEDKGEEETFPIPKAFVGNQLPFVGFTYYS
NRRYLSSANPNDNR.

Thus, the inventors have shown that the compounds of formula (I) of the invention act as agents dissociating the ROCK/PDK1 protein complex or preventing its formation in immune cells, by intercalating between the ROCK and PDK1 protein kinases at the contact surface between the two protein kinases, thus blocking the interaction between the two ROCK and PDK1 protein kinases. The compounds of formula (I) have broad-spectrum immunosuppressive action in the immune cells, acting both as an anti-inflammatory agent and as an anti-interferon agent, as shown by the examples of the present application.

The present invention also relates to a compound of formula (I) for its use in the prevention and/or treatment of a disease requiring the use of an inhibitor of the interaction between the ROCK protein kinase and the PDK1 protein kinase, said disease being selected from: inflammatory diseases, viral infections, bacterial infections and autoimmune diseases.

The present invention also relates to a pharmaceutical composition comprising the at least one compound of formula (I) according to the invention or a pharmaceutically acceptable salt thereof and at least one pharmaceutically acceptable excipient and/or support and/or a diluent and/or a pharmaceutically acceptable carrier.

Advantageously, the pharmaceutical composition further comprises at least a second active ingredient. Advantageously, the pharmaceutical composition is suitable for simultaneous administration or sequential administration. Advantageously, the pharmaceutical composition is in a form suitable for its administration by oral, parenteral or topical route.

The present invention also relates to a pharmaceutical composition comprising the at least one compound of formula (I) according to the invention or a pharmaceutically acceptable salt thereof and at least one pharmaceutically acceptable excipient and/or support and/or a diluent and/or a pharmaceutically acceptable carrier for its use in the prevention and/or treatment of a disease requiring the use of an inhibitor of the ROCK enzyme and/or of the PDK1 enzyme.

The present invention also relates to a pharmaceutical composition comprising the at least one compound of formula (I) according to the invention or a pharmaceutically acceptable salt thereof and at least one pharmaceutically acceptable excipient and/or support and/or a diluent and/or a pharmaceutically acceptable carrier for its use in the prevention and/or treatment of a disease requiring the use of an inhibitor of the interaction between the ROCK protein kinase and the PDK1 protein kinase, said disease being selected from: inflammatory diseases, viral infections, bacterial infections and autoimmune diseases.

### DETAILED DESCRIPTION

Within the meaning of the present, “inhibitor” is understood to mean a compound capable of inhibiting or blocking or neutralizing the expression of the targeted metabolic activity; the metabolic activity being weaker, or even zero in the presence of the inhibitor. Advantageously, the inhibitor of the interaction between the ROCK and PDK1 protein kinases is capable of blocking the interaction between the two proteins, either by competing with one of the protein kinases, or by blocking one of the protein kinases, thus causing a decrease in the expression of the activity of the ROCK and PDK1 protein kinases. Advantageously, the inhibitor of the interaction between the ROCK and PDK1 protein kinases is neutralizing. In particular, it inhibits or neutralizes the biological function of one of the ROCK or PDK1 protein kinases, which has the consequence of protecting the cells from inflammatory stress. The neutralization capacity of the inhibitor can be tested by a standard assay, especially by measuring the capacity of the inhibitor to block the interaction between the ROCK and PDK1 protein kinases and reduce the enzymatic activity of the ROCK and PDK1 kinases (IC₅₀). Advantageously, the inhibitor has an IC₅₀ of between 11 nM and 30 nM.

Advantageously, the inhibitor of the interaction between the ROCK and PDK1 protein kinases is an agent dissociating the ROCK-PDK1 protein kinase complex or preventing the formation of the ROCK-PDK1 protein kinase complex.

Within the meaning of the present, “agent dissociating the ROCK/PDK1 protein kinase complex or preventing the formation thereof” is understood to mean an intercalating agent capable of dissociating the ROCK-PDK1 protein kinase complex or an intercalating agent capable of preventing the formation of the ROCK/PDK1 protein kinase complex, fixing in the interaction zone between the ROCK and PDK1 protein kinases, said interaction zone being different from the active sites of the ROCK and PDK1 protein kinases. By fixing in the interaction zone between the ROCK and PDK1 protein kinases, the dissociating agent causes a steric clash in the immune cell, separating the ROCK protein kinase from the PDK1 protein kinase or preventing the interaction of the ROCK protein kinase with the PDK1 protein kinase. Advantageously, the compounds of formula (I) according to the invention are inhibitors of the interaction between the ROCK and PDK1 protein kinases, in particular the compounds of formula (I) according to the invention are agents dissociating the ROCK/PDK1 protein kinase complex.

Advantageously, the inventors have shown that the selective inhibition of the interaction between the ROCK and PDK1 protein kinases with one of the compounds of formula (I) according to the invention makes it possible to block the secretion of inflammatory cytokines, such as tumor necrosis factor alpha (TNFα), interleukins IL-1β, IL-6, IL-10, IL-12 p70, IL-18, IL-23, IL-33 but also the different subtypes of interferons (IFN), such as interferon alpha-2 (IFNα2), interferon gamma (IFNγ) and the MCP1 protein by the immune cells, showing a beneficial effect of the compounds of formula (I) according to the invention with respect to inflammatory stress without affecting the viability of said immune cells. Advantageously, the compounds of formula (I) according to the invention inhibit the secretion of inflammatory cytokines and interferons by an immune cell activated in vitro and ex vivo, especially when the compounds of formula (I) according to the invention are placed in contact with immune cells.

Within the meaning of the present invention, “immunity cell” or “immune system cell” or “immune cell” is understood to mean any cell of the immune system which originates from a hematopoietic stem cell in the bone marrow.

Advantageously, the immune cells according to the invention are peripheral blood mononuclear cells (PBMCs). The term “peripheral blood mononuclear cells (PBMCs)” refers to peripheral blood cells that have a round nucleus. These mononuclear blood cells recirculate between the tissues and the blood and are an essential element of the immune system for combating infections and adapting to intruders. There are two main types of mononuclear cells: lymphocyte cells and myeloid cells. The lymphocyte population of the PBMCs generally consists of T cells, B cells and NK cells. The myeloid population of the PBMCs consists of dendritic cells (myeloids and plasmacytoids) and monocytes/macrophages. The PBMCs can be isolated from whole blood samples by processes that are well known in the art (for example, Ficoll gradient). Advantageously, the immune cell is a T lymphocyte. Advantageously, the immune cell is a B lymphocyte.

Within the meaning of the present invention, the terms “T lymphocyte” and “T cell” are used interchangeably and refer to one of the main types of white blood cell, which completes its maturation in the thymus and which plays various roles in the immune system, including the identification of specific foreign antigens in the body and the activation and/or deactivation of other immune cells. The T lymphocyte may be any T cell selected from: a cultured T cell, for example, a primary T cell, or a T cell of a cultured T cell line, for example, Jurkat, SupTI, etc., or a T cell obtained from a mammal. The T cells may be CD3+ cells. The T cell may be any type of T cell and may be at any development stage, including, but not limited to, double-positive CD4+/CD8+ T cells, CD4+ helper T cells (for example, Th1 and Th2 cells), CD8+ cells (for example, cytotoxic T cells), a T cell present among the peripheral blood mononuclear cells (PBMCs), the peripheral blood leukocytes (PBLs), a T cell that is part of the tumor-infiltrating lymphocytes (TILs), memory T cells, naive T cells, regulatory T cells, gamma delta T cells (γδ T cells). Additional types of helper T cells comprise cells such as Th3, Th17, Th9 or Tfh cells. Additional types of memory T cells comprise cells such as central memory T cells (Tcm cells), effector memory T cells (Tem cells and TEMRA cells). T cell may also refer to a genetically modified T cell, such as a T cell modified to express a T cell receptor (TCR) or a chimeric antigen receptor (CAR). The T cell can also be differentiated from a stem cell or a progenitor cell.

Within the meaning of the present invention, “B cell” or “B lymphocyte cell” or “B lymphocyte” is understood to mean a lymphocytic cell which is produced in the bone marrow, which produces immunoglobulins and is involved in the production of antibodies during the humoral immune response. The B lymphocyte may be any B cell, selected from a B stem cell, a pro-B cell, a pre-B cell, a naive B cell, an activated B cell, a GC memory B cell, a late plasmablastic cell and a plasma cell.

Within the meaning of the present invention, the term “placing in contact”, when used with reference to an action carried out on an immune cell, is used interchangeably to denote culture, incubation or exposure of an immune cell with one or more of the inhibitors of the interaction between the ROCK and PDK1 protein kinases according to the invention. The cells placed in contact with a stimulation agent, such as R848, CpG-A, or placed in contact with another carrier are examples of cells placed in contact.

Within the meaning of the present invention, a cell that is “not placed in contact” or “untreated” is a cell that has not been treated, for example, cultured, placed in contact or incubated, with one or more of the inhibitors of the interaction between the ROCK and PDK1 protein kinases according to the invention.

Within the meaning of the present invention, “synthesis of inflammatory cytokines” or “production of inflammatory cytokines” or “secretion of inflammatory cytokines” is understood to mean the release of inflammatory cytokines into the external environment by a cell, advantageously an immune cell.

Within the meaning of the present invention, “interferon synthesis” or “interferon production” or “interferon secretion” is understood to mean the release of interferon into the external environment by a cell, advantageously an immune cell.

Within the meaning of the present invention, the “ROCK protein kinase” is a serine/threonine kinase, also referred to as RhoA-associated coiled-coil containing kinase or RhoA-Rho-associated kinase. Within the meaning of the present invention, the ROCK protein kinase may be isoform 1, referred to as ROCK1, or isoform 2, referred to as ROCK2. In one particularly advantageous embodiment of the invention, the ROCK protein kinase is the ROCK1 protein kinase.

Within the meaning of the present invention, “interaction zone between the ROCK and PDK1 protein kinases” is understood to mean a binding zone, different from the catalytic site (also referred to as active site) of each of the ROCK and PDK1 protein kinases, which makes it possible to associate the ROCK and PDK1 kinases into a single protein complex. Four interaction sites between the ROCK1 and PDK1 protein kinases were defined by modeling. The interaction between ROCK1 and PDK1 is based on weak bonds of the hydrogen or electrostatic type, among others. Advantageously, it may be an allosteric site of the ROCK1 protein kinase and/or of the PDK1 protein kinase.

The interaction site between the ROCK and PDK1 protein kinases targeted by the compounds of formula (I) according to the present invention relates to the following amino acids of the ROCK1 protein kinase: residues of proline 21 to glutamate 24 (P21-E24), tryptophan 122 to valine 136 (W122-V136), alanine 188 to phenylalanine 194 (A188-F194), lysine 222 (K222) and arginine 404 to tyrosine 405 (R404-Y405), the positions of the amino acids being established with respect to the amino acid sequences of the ROCK1 protein kinase (SEQ ID No. 11).

In other words, the compounds of formula (I) according to the present invention interact with the following amino acids: proline 21 to glutamate 24 (P21-E24), tryptophan 122 to valine 136 (W122-V136), alanine 188 to phenylalanine 194 (A188-F194), lysine 222 (K222) and arginine 404 to tyrosine 405 (R404-Y405) of the ROCK1 protein kinase, the positions of the amino acids being established with respect to the amino acid sequences of the ROCK1 protein kinase (SEQ ID No. 11).

As described hereinbefore, the compounds according to the invention correspond to the general formula (I):

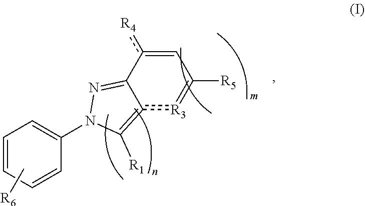

wherein:
m is selected from 0, 1 and 2,
n is selected from 0, 1 and 2,
R₁ is a —COR₂ group, wherein R₂ is selected from the group consisting of an —OH group, an —NH₂ group, an alkoxy group, an alkylamine group, an arylalkylamine group, an —NH—NH₂ group, and a —CH₂—Ar group optionally substituted by at least one halogen atom, wherein Ar is an aryl group,
R₃ is a nitrogen atom, an —NHCOR₇ group, an —NHCOOR₈ group, or a —COSR₉ group, wherein R₇, R₈ and R₉ are C₁ to C₆ alkyl groups optionally substituted by at least one halogen atom,
R₄ is a nitrogen atom or an —NH₂ group,
R₅ and R₆, which are identical or different, are selected independently from each other in the group consisting of a hydrogen atom, an —OH group, an —NH₂ group, an alkoxy group, a thiol group, a sulfonamide group, and an alkylamine group, and an aryl group optionally substituted by at least one halogen atom, and
   wherein the lines [symbol] and [symbol] , which are identical or different, independently of each other, depict a single covalent bond or a triple covalent bond, or a pharmaceutically acceptable salt thereof.

The term “pharmaceutically acceptable” means what is useful in the preparation of a generally safe, non-toxic pharmaceutical composition and neither biologically nor otherwise undesirable and comprises what is acceptable for veterinary and human pharmaceutical use.

The compounds of formula (I) according to the invention can be used in the form of a pharmaceutically acceptable salt and may comprise salts, hydrates and solvates prepared according to conventional processes, that are well known to a skilled person. By way of examples, mention may be made especially of the salts derived from carboxylic acids, such as the carboxylates (COO⁻), but also the sulfonates (—SO₃⁻), the phosphonates (PO₃²⁻), the quaternary ammoniums (NR₄⁺) or the sulfonamines (SO₂—NH₃⁺). Advantageously, the suitable salts comprise pharmaceutically or physiologically acceptable acid addition salts. Advantageously, the suitable salts comprise acid addition salts formed with various pharmaceutically or physiologically acceptable free acids. Examples of acids may comprise, without limitation, hydrochloric acid, bromic acid, sulfuric acid, phosphoric acid, citric acid, acetic acid, lactic acid, tartaric acid, fumaric acid, formic acid, propionic acid, oxalic acid, trifluoroacetic acid, methanesulfonic acid, benzenesulfonic acid, maleic acid, benzoic acid, gluconic acid, glycolic acid, succinic acid, 4-morpholineethanesulfonic acid, camphorsulfonic acid, nitrobenzenesulfonic acid, hydroxy-O-sulfonic acid, 4-toluenesulfonic acid, knife ruktu acid, EMBO acid, glutamic acid, aspartic acid, etc.

In addition, pharmaceutically acceptable metal salts may be prepared using bases. For example, alkali metal or alkaline earth metal salts may be obtained by dissolving the compound in an excess of an alkali metal hydroxide or alkaline earth metal hydroxide solution, filtering the undissolved compound salts and evaporating and drying the filtrate. Herein, the salts of sodium, potassium, lithium, cesium, magnesium or calcium are pharmaceutically suitable metal salts, without being limited thereto. In addition, silver salts corresponding to the metal salts may be obtained by reacting alkali metals or alkaline earth metals with suitable silver salts (for example, nitrates). Mention may also be made of ammoniums (NH4+) or amines in ammonium form, such as diethylamine (EDTA), pyrrolidine, piperidine and pyridine.

Within the meaning of the present invention, “a halogen atom” is understood to mean a fluorine, chlorine, bromine or iodine atom. The halogen atoms may more particularly be fluorine atoms.

Within the meaning of the present invention, “Cₜ-C_{z}” is understood to mean a carbon chain optionally containing from t to z carbon atoms, wherein t and z can take values from 1 to 10; for example, C₁-C₆ is a carbon chain that may contain 1 to 6 carbon atoms.

Within the meaning of the present invention, “alkyl” is understood to mean a linear or branched saturated aliphatic group, comprising especially from 1 to 6 carbon atoms. As examples, mention may be made of methyl, ethyl, n-propyl, isopropyl, butyl, isobutyl, tert-butyl, pentyl, neopentyl, etc. Advantageously, the alkyl may or may not be substituted by at least one halogen atom, advantageously at least two halogen atoms, advantageously at least three halogen atoms, advantageously at least four halogen atoms. In one particular embodiment of the invention, the alkyl may be substituted by at least one fluorine atom, advantageously at least two fluorine atoms, advantageously at least three fluorine atoms, advantageously at least four fluorine atoms.

Within the meaning of the present invention, “alkoxy” is understood to mean a linear or branched hydrocarbon chain comprising the specified number of carbon atoms, advantageously between 1 and 6 carbon atoms, and an oxygen atom. By way of examples, mention may be made especially of a methoxy group, or an ethoxy group, or a propoxy group or a butoxy group.

Within the meaning of the present invention, “alkylamine” is understood to mean a linear or branched hydrocarbon chain comprising the specified number of carbon atoms, advantageously between 1 and 6 carbon atoms, and a primary amine. By way of examples, mention may be made especially of a methylamine group, or an ethylamine group, or a propylamine group or a butylamine group.

Within the meaning of the present invention, “arylalkylamine” is understood to mean an alkylamine group substituted by one or more aryl groups, wherein the aryl group and the alkylamine group may or may not be supported by the same carbon atom. By way of examples of aryl groups, mention may be made of the group comprising phenylmethylamine, phenylethylamine, phenylpropylamine, (p-tolyl)methylamine, and the like.

Within the meaning of the present invention, “aryl” is understood to mean a monocyclic or bicyclic aromatic group containing between 5 and 10 carbon atoms, especially between 6 and 10 carbon atoms. By way of examples of aryl groups, mention may be made of the phenyl, tolyl, xylyl, mesityl or naphthyl group. Advantageously, the aryl group is phenyl. Advantageously, the aryl may or may not be substituted by at least one halogen atom, advantageously at least two halogen atoms, advantageously at least three halogen atoms, advantageously at least four halogen atoms. In one particular embodiment of the invention, the aryl may be substituted by at least one fluorine atom, advantageously at least two fluorine atoms, advantageously at least three fluorine atoms, advantageously at least four fluorine atoms. In another embodiment, the aryl groups may comprise one or more heteroatoms, selected from N, O and/or S, advantageously 1 to 4 heteroatoms. By way of example of an aryl group comprising one or more heteroatoms, mention may be made especially of pyridine, furan, thiophene, pyrrole, pyrazole, imidazole, thiazole, isoxazole, isothiazole, pyridazine, pyrimidine, pyrazine, triazines, indolizine, indole, isoindole, benzofuran, benzothiophene, indazole, benzimidazole, benzothiazole, purine, quinoline, isoquinoline, cinnoline, phthalazine, quinazoline, quinoxaline, pteridine, naphthyridines, carbazole, phenothiazine, phenoxazine, acridine, phenazine, oxazole, pyrazole, oxadiazole, triazole, thiadiazole and the unsaturated derivatives thereof. Other examples are the unsaturated derivatives of pyrrolidine, dioxolane, imidazolidine, pyrazolidine, piperidine, dioxane, morpholine, dithiane, thiomorpholine, piperazine and trithiane.

According to the present invention, R₁ is a —COR₂ group, wherein R₂ is selected from the group consisting of an —OH group, an —NH₂ group, an alkoxy group, an alkylamine group, an arylalkylamine group, an —NH—NH₂ group, and a —CH₂—Ar group optionally substituted by at least one halogen atom, wherein Ar is an aryl group. Advantageously, the aryl group is a phenyl group.

Advantageously, R₁ is selected from:

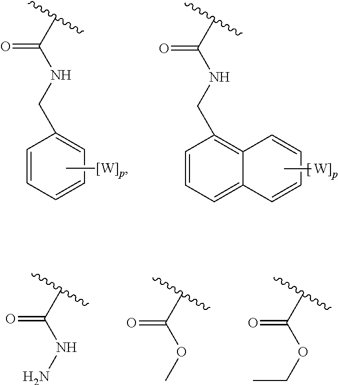

wherein W is a halogen atom, identical or different, advantageously selected from a fluorine, chlorine, bromine or iodine atom, advantageously a fluorine atom, or a C₁ to C₆ alkyl, wherein p is 0, 1, 2, 3, 4 or 5, and wherein q is 0, 1, 2, 3, 4, 5, 6 or 7.

Advantageously, R₁ is:

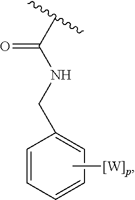

wherein p is 0.

Advantageously, R₁ is:

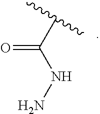 [SMILES: *C(=O)NN]

Advantageously, R₁ is:

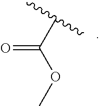 [SMILES: *C(=O)OC]

According to the present invention, R₃ is a nitrogen atom, an —NHCOR₇ group, an —NHCOOR₈ group, or a —COSR₉ group, wherein R₇, R₈ and R₉ are C₁ to C₆ alkyl groups optionally substituted by at least one halogen atom. Advantageously, R₃ is a nitrogen atom. Advantageously, R₃ is an —NHCOR₇ group, wherein R₇ is a C₁ to C₆ alkyl group optionally substituted by at least one halogen atom.

According to one particular embodiment of the invention, R₃ is a nitrogen atom, when the bond [symbol] depicts a triple covalent bond.

According to one particular embodiment of the invention, when the bond [symbol] depicts a single covalent bond, R₃ can be selected from a nitrogen atom, an —NHCOR₇ group, an —NHCOOR₈ group, or a —COSR₉ group, wherein R₇, R₈ and R₉ are C₁ to C₆ alkyl groups optionally substituted by at least one halogen atom.

Advantageously, the —NHCOR₇ group is selected from:

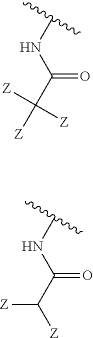

wherein Z is a halogen atom, selected from a fluorine, chlorine, bromine or iodine atom, advantageously a fluorine atom, or a C₁ to C₆ alkyl group.

According to the present invention, R₄ is a nitrogen atom or an —NH₂ group.

Advantageously, R₄ is a nitrogen atom, when the bond [symbol] depicts a triple covalent bond.

Advantageously, R₄ is an —NH₂ group, when the bond [symbol] depicts a single covalent bond.

According to the present invention, R₅ is selected from the group consisting of a hydrogen atom, an —OH group, an —NH₂ group, an alkoxy group, a thiol group, a sulfonamide group, an alkylamine group, and an aryl group optionally substituted by at least one halogen atom. Advantageously, R₅ is an aryl group optionally substituted by at least one halogen atom, advantageously a phenyl group optionally substituted by at least one halogen atom, advantageously an unsubstituted phenyl group.

According to the present invention, R₆ is selected from the group consisting of a hydrogen atom, an —OH group, an —NH₂ group, an alkoxy group, a thiol group, a sulfonamide group, an alkylamine group, and an aryl group optionally substituted by at least one halogen atom. Advantageously, R₆ is an alkoxy group, even more advantageously, R₆ is a methoxy group. In one particular embodiment of the invention, the R₆ group can be in position 3, 4 or 5 on the ring.

In one particular embodiment of the invention, the compounds of formula (I) according to the invention or a pharmaceutically acceptable salt thereof comprise compounds, wherein m is 0 and n is 0 and wherein the bonds [symbol] and [symbol] depict a triple covalent bond. Advantageously, the compounds of general formula (I) wherein m is 0 and n is 0 and wherein the bonds [symbol] and [symbol] depict a triple covalent bond are represented by the subgroup of compounds of formula (II) or a pharmaceutically acceptable salt thereof,

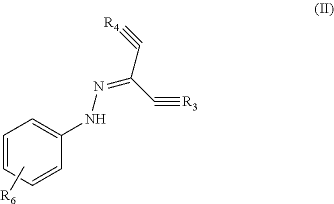

wherein R₃, R₄ and R₆ are as defined in formula (I).

By way of examples of compounds of formula (II), mention may be made especially of the compounds:

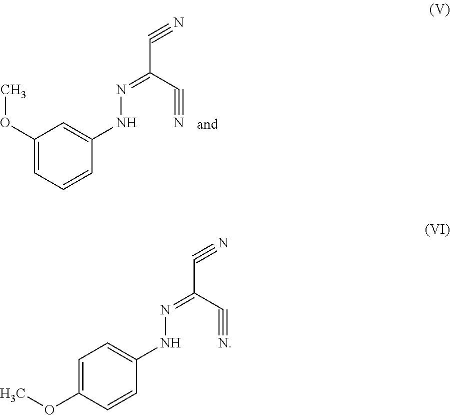

In another particular embodiment of the invention, the compounds of formula (I) according to the invention or a pharmaceutically acceptable salt thereof comprise compounds wherein m is 1 and n is 1 and wherein the bonds [symbol] and [symbol] depict a single covalent bond.

Advantageously, the compounds of general formula (I) wherein m is 1 and n is 1 and wherein the bonds [symbol] and [symbol] depict a single covalent bond are represented by the subgroup of compounds of formula (III) or a pharmaceutically acceptable salt thereof,

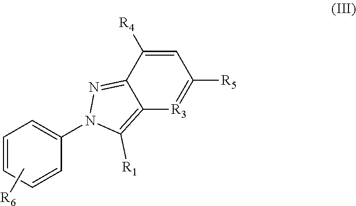

wherein R₃, R₄, R₅ and R₆ are as defined in formula (I).

By way of examples of compounds of formula (III), mention may be made especially of the compounds:

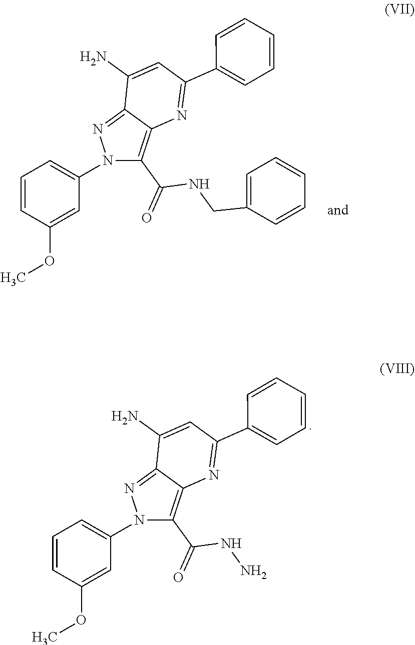 [SMILES: COc1cccc(-n2nc3c(N)cc(-c4ccccc4)nc3c2C(=O)NCc2ccccc2)c1 ; COc1cccc(-n2nc3c(N)cc(-c4ccccc4)nc3c2C(=O)NN)c1]

In another particular embodiment of the invention, the compounds of formula (I) according to the invention or a pharmaceutically acceptable salt thereof comprise compounds wherein m is 1 and n is 1 and wherein the bonds [symbol] and [symbol] depict a single covalent bond.

Advantageously, the compounds of general formula (I) wherein m is 0 and n is 1 and wherein the bonds [symbol] and [symbol] depict a single covalent bond are represented by the subgroup of compounds of formula (IV) or a pharmaceutically acceptable salt thereof,

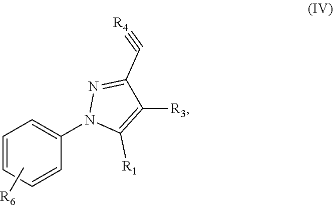

wherein R₁, R₃, R₄ and R₆ are as defined in formula (I).

By way of examples of compounds of formula (IV), mention may be made especially of the compounds:

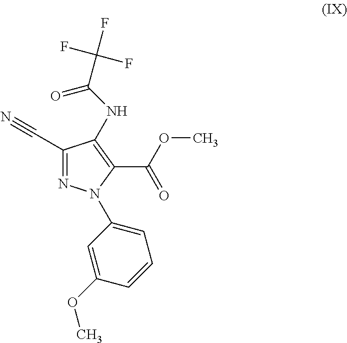 [SMILES: COC(=O)c1c(NC(=O)C(F)(F)F)c(C#N)nn1-c1cccc(OC)c1]

In one particularly advantageous embodiment of the invention, the compounds of general formula (I) are selected from:

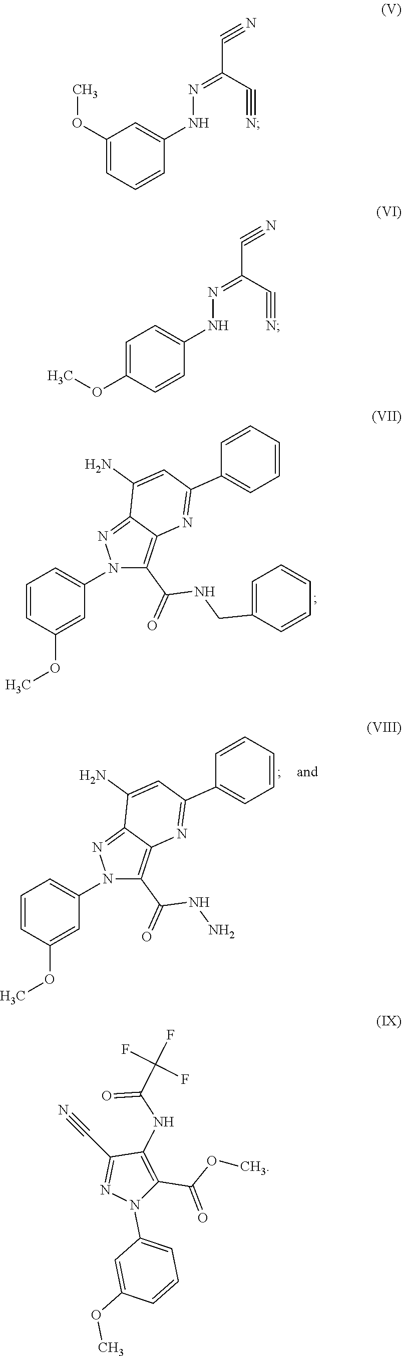

Advantageously, the inventors have shown that the incubation of human peripheral blood mononuclear cells with a compound of formula (I) and in particular with the compound of formula (V), before being stimulated with an agonist (R848) of the toll-like receptors (TLR) 7/8 to mimic an infection with a single-stranded RNA virus, inhibits the production of pro-inflammatory cytokines, such as IL-1β, IL-6, IL-10, IL-12 p70, IL-18, IL-23, IL-33, TNFα and the NF-κB protein, but also the different subtypes of interferons (IFN), such as interferon alpha-2 (IFNα2), interferon gamma (IFNγ), as well as the MCP1 protein, induced by the TLR7/8 stimulation.

Advantageously, the inventors have also shown that the incubation of human peripheral blood mononuclear cells with a compound of formula (I) and in particular with the compound of formula (VI), before being stimulated with an agonist (R848) of the toll-like receptors (TLR) 7/8 to mimic an infection with a single-stranded RNA virus, inhibits the production of pro-inflammatory cytokines, such as IL-1β, IL-6, IL-10, IL-12 p70, IL-18, IL-23, IL-33, TNFα and the MCP1 protein, but also the different subtypes of interferons (IFN), such as interferon alpha-2 (IFNα2), interferon gamma (IFNγ), as well as the MCP1 protein, induced by the TLR7/8 stimulation.

Advantageously, the inventors have also shown that the incubation of human peripheral blood mononuclear cells with a compound of formula (I) and in particular with the compounds of formula (VII), of formula (VIII) and of formula (IX), before being stimulated with an agonist (R848) of the toll-like receptors (TLR) 7/8 to mimic an infection with a single-stranded RNA virus, inhibits the production of interferons (IFN), induced by the TLR7/8 stimulation.

Advantageously, the inventors have also shown that the incubation of human peripheral blood mononuclear cells with a compound of formula (I) and in particular with the compound of formula (V) or the compound of formula (VI), before being stimulated with an agonist (LPS) of the toll-like receptors (TLR) 4 to mimic a bacterial infection, inhibits the production of TNFα induced by the TLR4 stimulation, but also the secretion of pro-inflammatory cytokines on human peripheral blood mononuclear cells, such as IL-6, IL-1β, IL-18, IL-23, MCP1 and the interferons, in particular interferon gamma (IFNγ) induced by the stimulation of TLR4 by lipopolysaccharide (LPS), LPS being a bacterial agent inducing an inflammatory response.

Advantageously, the inventors have also shown that the compounds of formula (V) to (VIII) are capable of blocking the activation of the B lymphocytes from healthy donors in response to stimulation by toll-like receptors TLR9.

Advantageously, the inventors have also shown that the compounds of formula (V) and (VI) are capable of:
preventively blocking the production of TNFα on monocytes isolated from peripheral blood mononuclear cells (PBMCs) from healthy donors, to be stimulated with an agonist (R848) of the toll-like receptors (TLR) 7/8 to mimic an infection with a single-stranded RNA virus;
preventively blocking the production of TNFα on monocytes isolated from peripheral blood mononuclear cells (PBMCs) from healthy donors, to be stimulated with an agonist (LPS) of the toll-like receptors (TLR) 4 to mimic a gram-negative bacterial infection;
preventively blocking the production of TNFα on monocytes isolated from peripheral blood mononuclear cells (PBMCs) from healthy donors, to be stimulated with an agonist (LTA: lipoteichoic acid) of the toll-like receptors (TLR) 2 to mimic a gram-positive bacterial infection;
preventively blocking the production of TNFα on plasmacytoid dendritic cells isolated from peripheral blood mononuclear cells (PBMCs) from healthy donors, to be stimulated with an activator (R848) of the toll-like receptors (TLR) 7/8 to mimic an infection with a single-stranded RNA virus;
preventively blocking the production of TNFα on plasmacytoid dendritic cells isolated from peripheral blood mononuclear cells (PBMCs) from healthy donors, to be stimulated with an agonist (CpG-A) of the toll-like receptors (TLR) 9 capable of recognizing viral or bacterial DNA rich in CpG units.

Thus, the inventors have shown quite surprisingly that the use of a compound of formula (I) has a preventive effect on the inhibition of the production of inflammatory cytokines induced by the stimulation of TLR2 or TLR4 or TLR7/8 or TLR9 regardless of the immune cell.

Thus, the inventors have shown quite surprisingly that the use of a compound of formula (I) has a curative effect on interferon production induced by the stimulation of TLR2 or TLR4 or TLR7/8 or TLR9 regardless of the immune cell.

The preferred compound of formula (I) is one of the compounds mentioned in Table 1 hereunder.

TABLE 1

| Structure | Compound name |
|---|---|
| 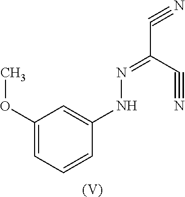 | N-(3-methoxyphenyl)carbonohydrazonoyl dicyanide) {IUPAC Name: 2-[(3- methoxyphenyl)hydrazinylidene] propanedinitrile} |
| 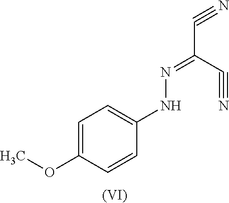 | N-(4-methoxyphenyl)carbonohydrazonoyl dicyanide) {IUPAC name: 2-[(4- methoxyphenyl)hydrazinylidene] propanedinitrile} |
| 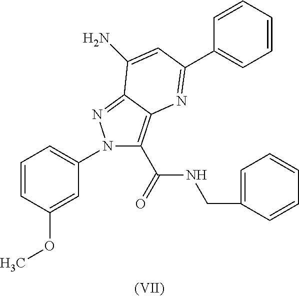 [SMILES: COc1cccc(-n2nc3c(N)cc(-c4ccccc4)nc3c2C(=O)NCc2ccccc2)c1 ; "(VII)"] | 7-amino-N-benzyl-2-(3-methoxyphenyl)-5- phenyl-2H-pyrazolo[4,3-b]pyridine-3- carboxamide |
| 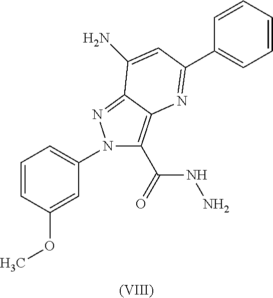 | 7-amino-2-(3-methoxyphenyl)-5-phenyl-2H- pyrazolo[4,3-b]pyridine-3-carbohydrazide |
| 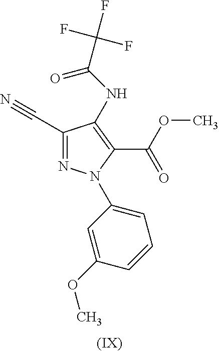 | methyl 3-cyano-1-(3-methoxyphenyl)-4- (2,2,2-trifluoroacetamido)-1H-pyrazole-5- carboxylate |

Advantageously, the compound of formula (I) is selected from: 2-[(3-methoxyphenyl)hydrazinylidene]propanedinitrile; 2-[(4-methoxyphenyl)hydrazinylidene]propanedinitrile; 7-amino-N-benzyl-2-(3-methoxyphenyl)-5-phenyl-2H-pyrazolo[4,3-b]pyridine-3-carboxamide; 7-amino-2-(3-methoxyphenyl)-5-phenyl-2H-pyrazolo[4,3-b]pyridine-3-carbohydrazide and methyl 3-cyano-1-(3-methoxyphenyl)-4-(2,2,2-trifluoroacetamido)-1H-pyrazole-5-carboxylate

Another aspect of the present invention relates to a compound of formula (I) for its use as a drug, in particular a drug intended to block and/or inhibit the interaction between the ROCK and PDK1 protein kinases. Advantageously, the compound of formula (I) is capable of intercalating at the interaction zone between the two ROCK and PDK1 protein kinases, thus causing either a dissociation of the ROCK/PDK1 protein kinase complex, or preventing the formation of the ROCK and PDK1 protein kinase complex. Advantageously, the compounds of formula (I) according to the invention are agents dissociating the ROCK and PDK1 protein kinase complex. Advantageously, the compounds of formula (I) according to the invention are agents preventing the formation of the ROCK and PDK1 protein kinase complex.

According to one particular embodiment, the compounds of formula (I) according to the invention are anti-inflammatory and anti-interferon agents.

Within the meaning of the present invention, “anti-inflammatory and anti-interferon agent” is understood to mean an agent capable of preventing and/or blocking and/or inhibiting the synthesis of inflammatory cytokines and interferons and in particular an agent capable of preventing and/or blocking and/or inhibiting the secretion of at least one inflammatory cytokine, such as tumor necrosis factor alpha (TNFα), interleukins IL-1β, IL-6, IL-10, IL-12p70, IL-18, IL-23, IL-33 and the MCP1 protein, but also at least one subtype of interferons (IFN), such as interferon alpha-2 (IFNα2), interferon gamma (IFNγ) by an immune cell, when said agent is placed in contact with an immune cell.

Advantageously, the compounds of formula (I) may be used in the prevention and/or treatment of a disease requiring the use of an inhibitor of the interaction between the ROCK protein kinase and the PDK1 protein kinase.

In other words, the present invention relates to the use of a compound of formula (I) for the preparation of a drug, in particular a drug intended to block and/or inhibit the interaction between the ROCK and PDK1 protein kinases.

In other words, the present invention relates to the preparation of a drug comprising a compound of formula (I) for blocking and/or inhibiting the interaction between the ROCK and PDK1 protein kinases.

Advantageously, said drug may be used in the prevention and/or treatment of a disease requiring the use of an inhibitor of the interaction between the ROCK protein kinase and the PDK1 protein kinase.

Advantageously, the compound of formula (I) is particularly effective in the prevention and/or treatment of a disease requiring the use of an inhibitor of the interaction between the ROCK protein kinase and the PDK1 protein kinase.

Advantageously, the compound of formula (I) is particularly effective in the prevention and/or treatment of a disease requiring the use of an inhibitor of the interaction between the ROCK protein kinase and the PDK1 protein kinase selected from the inflammatory diseases, viral infections, bacterial infections and autoimmune diseases.

Another aspect of the present invention relates to a pharmaceutical composition comprising at least one compound of formula (I) as previously described or an acceptable pharmaceutical salt thereof, as an active ingredient and at least one pharmaceutically acceptable excipient and/or a support and/or a diluent and/or a pharmaceutically acceptable carrier. Advantageously, at least one compound of formula (I) is an agent dissociating the ROCK/PDK1 protein kinase complex.

Advantageously, the pharmaceutical composition comprising at least one compound of formula (I) as previously described or an acceptable pharmaceutical salt thereof, as an active ingredient and at least one pharmaceutically acceptable excipient and/or a support and/or a diluent and/or a pharmaceutically acceptable carrier, is particularly effective in the prevention and/or treatment of a disease requiring the use of an inhibitor of the interaction between the ROCK protein kinase and the PDK1 protein kinase.

Advantageously, said pharmaceutical composition is particularly effective in the prevention and/or treatment of a disease requiring the use of an inhibitor of the interaction between the ROCK protein kinase and the PDK1 protein kinase selected from the inflammatory diseases, viral infections, bacterial infections and autoimmune diseases.

In one particular embodiment, the pharmaceutical composition comprises at least one compound of formula (I) as previously described or an acceptable pharmaceutical salt thereof, as an active ingredient and at least one pharmaceutically acceptable excipient and/or a support and/or a diluent and/or a pharmaceutically acceptable carrier. Advantageously, the pharmaceutical composition according to the invention may comprise a combination of at least two different compounds of formula (I), advantageously at least three different compounds of formula (I), advantageously at least four different compounds of formula (I), advantageously at least five different compounds of formula (I), advantageously at least six different compounds of formula (I), or more.

In one particular embodiment, the invention relates to a pharmaceutical composition comprising a pharmaceutically active amount of at least one compound of formula (I) or an acceptable pharmaceutical salt thereof as previously described and at least one pharmaceutically acceptable excipient and/or a support and/or a diluent and/or a pharmaceutically acceptable carrier.

Advantageously, the pharmaceutical composition according to the invention may comprise a combination of at least two different compounds of formula (I), advantageously at least three different compounds of formula (I), advantageously at least four different compounds of formula (I), advantageously at least five different compounds of formula (I), advantageously at least six different compounds of formula (I), or more.

In one embodiment according to the invention, the patient can be a human. In another embodiment of the invention, the patient can be a non-human animal, in particular a dog, a cat, a horse, a cow, a pig, a sheep, a goat, a deer or a primate.

According to embodiments that involve administering, to a patient requiring treatment, a therapeutically effective amount of at least one compound of formula (I) or an acceptable pharmaceutical salt thereof according to the invention, as defined herein, “therapeutically effective” or “an amount effective for treating” or “pharmaceutically effective”, the amount of compound of formula (I) or an acceptable pharmaceutical salt thereof or a composition necessary for inhibiting or reversing a disease (for example, for treating inflammation, viral infections and autoimmune diseases). The determination of a therapeutically effective amount depends specifically on factors such as the toxicity and efficacy of the drug. These factors will differ based on other factors such as power, relative bioavailability, body weight of the patient, seriousness of the unwanted effects and preferred mode of administration. Toxicity can be determined using processes that are well known in the art. Efficacy can be determined using the same indications. Efficacy can be measured by a decrease in inflammation or infection, for example, in a model of idiopathic juvenile arthritis. A pharmaceutically effective amount is therefore an amount that the clinician considers to be tolerable in toxicological terms, but effective.

The dosage can be adjusted appropriately to obtain the desired drug (for example, an inhibitor of the interaction between the ROCK and PDK1 protein kinases of the invention) at local or systemic levels, based on the mode of administration. In the case where the response in a patient is insufficient at such doses, even higher doses (or higher effective doses by a different and more localized administration route) can be used to the extent that the patient's tolerance allows it. Multiple doses per day may also be used to achieve suitable systemic levels of the compound of formula (I) or an acceptable pharmaceutical salt thereof according to the invention. Suitable systemic levels can be determined, for example, by measuring the patient's maximum or prolonged plasmatic level. “Dose” and “dosage” are used interchangeably herein.

In one advantageous embodiment of the invention, the amount of compound of formula (I) or an acceptable pharmaceutical salt thereof according to the invention or of a pharmaceutical composition comprising at least one compound of formula (I) or an acceptable pharmaceutical salt thereof as an active ingredient administered to a patient is 0.5 mg to 30 mg per kg of body weight, based on the intensity of the inflammation. Advantageously, the amount of compound of formula (I) or an acceptable pharmaceutical salt thereof according to the invention or of a pharmaceutical composition comprising at least one compound of formula (I) or an acceptable pharmaceutical salt thereof as an active ingredient administered to a patient is 1.0 mg, advantageously 1.5 mg, advantageously 2.0 mg, advantageously 2.5 mg, advantageously 3.0 mg, advantageously 3.5 mg, advantageously 4.0 mg, advantageously 4.5 mg, advantageously 5.0 mg, advantageously 5.5 mg, advantageously 6.0 mg, advantageously 6.5 mg, advantageously 7.0 mg, advantageously 7.5 mg, advantageously 8.0 mg, advantageously 8.5 mg, advantageously 9.0 mg, advantageously 9.5 mg, advantageously 10.0 mg, advantageously 10.5 mg, advantageously 11.0 mg, advantageously 11.5 mg, advantageously 12.0 mg, advantageously 12.5 mg, advantageously 13.0 mg, advantageously 13.5 mg, advantageously 14.0 mg, advantageously 14.5 mg, advantageously 15.0 mg, advantageously 15.5 mg, advantageously 16.0 mg, advantageously 16.5 mg, advantageously 17.0 mg, advantageously 17.5 mg, advantageously 18.0 mg, advantageously 18.5 mg, advantageously 19.0 mg, advantageously 19.5 mg, advantageously 20.0 mg, advantageously 20.5 mg, advantageously 21.0 mg, advantageously 21.5 mg, advantageously 22.0 mg, advantageously 22.5 mg, advantageously 23.0 mg, advantageously 23.5 mg, advantageously 24.0 mg, advantageously 24.5 mg, advantageously 25.0 mg, advantageously 25.5 mg, advantageously 26.0 mg, advantageously 26.5 mg, advantageously 27.0 mg, advantageously 27.5 mg, advantageously 28.0 mg, advantageously 28.5 mg, advantageously 29.0 mg, advantageously 29.5 mg, advantageously 30.0 mg per kg of body weight, based on the intensity of the inflammation.

Advantageously, the amount of compound of formula (I) or an acceptable pharmaceutical salt thereof according to the invention or of a pharmaceutical composition comprising at least one compound of formula (I) or an acceptable pharmaceutical salt thereof as an active ingredient administered to a patient is between 0.5 mg and 30.0 mg per kg of body weight, advantageously between 0.5 mg and 29.0 mg per kg of body weight, advantageously between 0.5 mg and 28.0 mg per kg of body weight, advantageously between 0.5 mg and 27.0 mg per kg of body weight, advantageously between 0.5 mg and 26.0 mg per kg of body weight, advantageously between 0.5 mg and 25.0 mg per kg of body weight, advantageously between 0.5 mg and 24.0 mg per kg of body weight, advantageously between 0.5 mg and 23.0 mg per kg of body weight, advantageously between 0.5 mg and 22.0 mg per kg of body weight, advantageously between 0.5 mg and 21 mg per kg of body weight, advantageously between 0.5 mg and 20.0 mg per kg of body weight, advantageously between 0.5 mg and 19.0 mg per kg of body weight, advantageously between 0.5 mg and 18.0 mg per kg of body weight, advantageously between 0.5 mg and 17.0 mg per kg of body weight, advantageously between 0.5 mg and 16.0 mg per kg of body weight advantageously between 0.5 mg and 15.0 mg per kg of body weight, advantageously between 0.5 mg and 14.0 mg per kg of body weight, advantageously between 0.5 mg and 13.0 mg per kg of body weight, advantageously between 0.5 mg and 12.0 mg per kg of body weight, advantageously between 0.5 mg and 11.0 mg per kg of body weight, advantageously between 0.5 mg and 10.0 mg per kg of body weight, advantageously between 0.5 mg and 9.0 mg per kg of body weight, advantageously between 0.5 mg and 8.0 mg per kg of body weight, advantageously between 0.5 mg and 7.0 mg per kg of body weight, advantageously between 0.5 mg and 6.0 mg per kg of body weight, advantageously between 1.0 mg and 6.0 mg per kg of body weight, advantageously between 1.5 mg and 6.0 mg per kg of body weight, advantageously between 1.5 mg and 5.5 mg per kg of body weight, advantageously between 2.0 mg and 5.0 mg per kg of body weight, based on the intensity of the inflammation.

In one advantageous embodiment of the invention, the amount of compound of formula (I) or an acceptable pharmaceutical salt thereof according to the invention or of a pharmaceutical composition comprising at least one compound of formula (I) or an acceptable pharmaceutical salt thereof as an active ingredient administered to a patient is from 0.5 mg to 30 mg per kg of body weight, based on the intensity of the infection. Advantageously, the amount of compound of formula (I) or an acceptable pharmaceutical salt thereof according to the invention or of a pharmaceutical composition comprising at least one compound of formula (I) or an acceptable pharmaceutical salt thereof as an active ingredient administered to a patient is 1.0 mg, advantageously 1.5 mg, advantageously 2.0 mg, advantageously 2.5 mg, advantageously 3.0 mg, advantageously 3.5 mg, advantageously 4.0 mg, advantageously 4.5 mg, advantageously 5.0 mg, advantageously 5.5 mg, advantageously 6.0 mg, advantageously 6.5 mg, advantageously 7.0 mg, advantageously 7.5 mg, advantageously 8.0 mg, advantageously 8.5 mg, advantageously 9.0 mg, advantageously 9.5 mg, advantageously 10.0 mg, advantageously 10.5 mg, advantageously 11.0 mg, advantageously 11.5 mg, advantageously 12.0 mg, advantageously 12.5 mg, advantageously 13.0 mg, advantageously 13.5 mg, advantageously 14.0 mg, advantageously 14.5 mg, advantageously 15.0 mg, advantageously 15.5 mg, advantageously 16.0 mg, advantageously 16.5 mg, advantageously 17.0 mg, advantageously 17.5 mg, advantageously 18.0 mg, advantageously 18.5 mg, advantageously 19.0 mg, advantageously 19.5 mg, advantageously 20.0 mg, advantageously 20.5 mg, advantageously 21.0 mg, advantageously 21.5 mg, advantageously 22.0 mg, advantageously 22.5 mg, advantageously 23.0 mg, advantageously 23.5 mg, advantageously 24.0 mg, advantageously 24.5 mg, advantageously 25.0 mg, advantageously 25.5 mg, advantageously 26.0 mg, advantageously 26.5 mg, advantageously 27.0 mg, advantageously 27.5 mg, advantageously 28.0 mg, advantageously 28.5 mg, advantageously 29.0 mg, advantageously 29.5 mg, advantageously 30.0 mg per kg of body weight, based on the intensity of the infection.

Advantageously, the amount of compound of formula (I) or an acceptable pharmaceutical salt thereof according to the invention or of a pharmaceutical composition comprising at least one compound of formula (I) or an acceptable pharmaceutical salt thereof as an active ingredient administered to a patient is between 0.5 mg and 30.0 mg per kg of body weight, advantageously between 0.5 mg and 29.0 mg per kg of body weight, advantageously between 0.5 mg and 28.0 mg per kg of body weight, advantageously between 0.5 mg and 27.0 mg per kg of body weight, advantageously between 0.5 mg and 26.0 mg per kg of body weight, advantageously between 0.5 mg and 25.0 mg per kg of body weight, advantageously between 0.5 mg and 24.0 mg per kg of body weight, advantageously between 0.5 mg and 23.0 mg per kg of body weight, advantageously between 0.5 mg and 22.0 mg per kg of body weight, advantageously between 0.5 mg and 21 mg per kg of body weight, advantageously between 0.5 mg and 20.0 mg per kg of body weight, advantageously between 0.5 mg and 19.0 mg per kg of body weight, advantageously between 0.5 mg and 18.0 mg per kg of body weight, advantageously between 0.5 mg and 17.0 mg per kg of body weight, advantageously between 0.5 mg and 16.0 mg per kg of body weight advantageously between 0.5 mg and 15.0 mg per kg of body weight, advantageously between 0.5 mg and 14.0 mg per kg of body weight, advantageously between 0.5 mg and 13.0 mg per kg of body weight, advantageously between 0.5 mg and 12.0 mg per kg of body weight, advantageously between 0.5 mg and 11.0 mg per kg of body weight, advantageously between 0.5 mg and 10.0 mg per kg of body weight, advantageously between 0.5 mg and 9.0 mg per kg of body weight, advantageously between 0.5 mg and 8.0 mg per kg of body weight, advantageously between 0.5 mg and 7.0 mg per kg of body weight, advantageously between 0.5 mg and 6.0 mg per kg of body weight, advantageously between 1.0 mg and 6.0 mg per kg of body weight, advantageously between 1.5 mg and 6.0 mg per kg of body weight, advantageously between 1.5 mg and 5.5 mg per kg of body weight, advantageously between 2.0 mg and 5.0 mg per kg of body weight, based on the intensity of the infection.

Of course, this unit dose can be repeated if necessary.

In one advantageous embodiment, the compound of formula (I) or an acceptable pharmaceutical salt thereof as an active ingredient is administered to the patient at a rate of 100 mg to 500 mg per day. Advantageously, the compound of formula (I) or an acceptable pharmaceutical salt thereof as an active ingredient is administered to the patient at a rate of 100 mg to 450 mg per day, advantageously at a rate of 100 mg to 400 mg per day, advantageously at a rate of 100 mg to 350 mg per day, advantageously at a rate of 100 mg to 300 mg per day, advantageously at a rate of 100 mg to 250 mg per day, advantageously at a rate of 100 mg to 200 mg per day, advantageously at a rate of 100 mg to 150 mg per day, advantageously at a rate of 140 mg per day. Of course, this unit dose can be repeated if necessary.

In one advantageous embodiment, the pharmaceutical composition according to the invention is administered to the patient at a rate of one, two, three, four, five, six or seven times a week.

Advantageously, the pharmaceutical composition according to the invention is administered to the patient at a rate of seven times per week, or one administration per day for seven consecutive days. Advantageously, the pharmaceutical composition according to the invention is administered to the patient at a rate of six times per week. Advantageously, the pharmaceutical composition according to the invention is administered to the patient at a rate of five times per week. Advantageously, the pharmaceutical composition according to the invention is administered to the patient at a rate of four times per week. Advantageously, the pharmaceutical composition according to the invention is administered to the patient at a rate of three times a week. Advantageously, the pharmaceutical composition according to the invention is administered to the patient at a rate of twice a week. Advantageously, the pharmaceutical composition according to the invention is administered to the patient at a rate of once a week.

In one advantageous embodiment of the invention, the provided pharmaceutical compositions are used for in vivo applications. Based on the intended mode of in vivo administration, the pharmaceutical compositions used may be in the solid, semi-solid or liquid dosage form such as, for example, tablets, pills, powders, capsules, gels, ointments, liquids, suspensions. Preferably, the pharmaceutical compositions are administered in unit dosage forms suitable for a single administration of precise doses. The pharmaceutical compositions may also comprise, based on the desired formulation, at least one pharmaceutically acceptable support or diluent, which are defined as aqueous-based carriers commonly used to formulate pharmaceutical compositions for animal or human administration. The diluent is chosen so as not to affect the biological activity of the compound of formula (I) or an acceptable pharmaceutical salt thereof according to the invention. Examples of such diluents are distilled water, physiological saline, Ringer's solution, a dextrose solution and Hank's solution. In addition, the pharmaceutical composition may also comprise other medicinal agents, pharmaceutical agents, supports, adjuvants, non-toxic, non-therapeutic, non-immunogenic stabilizers, etc. The effective amounts of such a diluent or carrier are effective amounts for obtaining a pharmaceutically acceptable solution in terms of formulation, solubility of components, biological activity, etc. In some embodiments, the pharmaceutical compositions provided herein are sterile.

It is possible to envisage formulating at least one compound of formula (I) or an acceptable pharmaceutical salt thereof according to the present invention at a rate of 0.5 to 98% by weight, expressed by weight, or even more, with respect to the total weight of the pharmaceutical composition under consideration.

In one particularly advantageous embodiment of the present invention, the pharmaceutical composition according to the invention comprises only at least one compound of formula (I) or an acceptable pharmaceutical salt thereof, as single active ingredient.

In one particular embodiment of the invention, the compound of general formula (I) of the pharmaceutical composition is selected from the compounds of formula (V) to (IX):

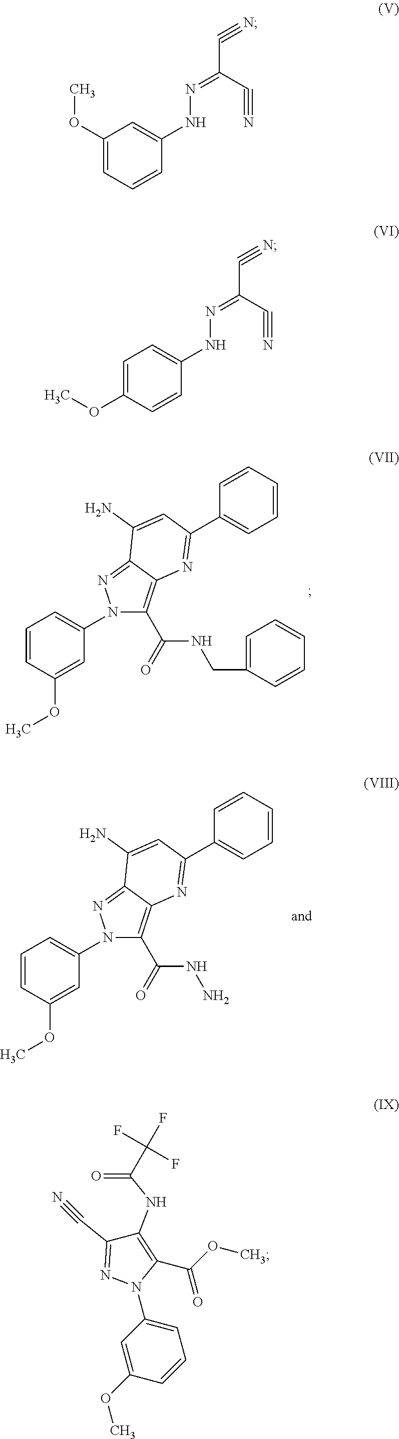

or one of the acceptable pharmaceutical salts thereof.

In one particularly advantageous embodiment of the invention, the pharmaceutical composition comprises the compound of formula (I) as previously defined, as single active ingredient

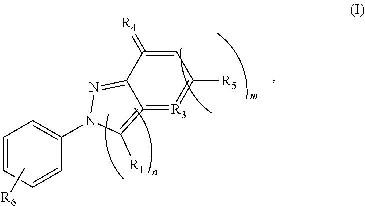

wherein m, n, R₁, R₂, R₃, R₄, R₅ and R₆ are as previously defined, or one of its acceptable pharmaceutical salts and at least one pharmaceutically acceptable excipient and/or a support or a diluent or a pharmaceutically acceptable carrier.

In one particularly advantageous embodiment of the invention, the pharmaceutical composition comprises the compound of formula (V) as previously defined, as single active ingredient

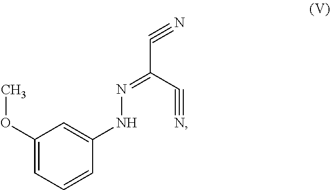

or one of its acceptable pharmaceutical salts and at least one pharmaceutically acceptable excipient and/or a support or a diluent or a pharmaceutically acceptable carrier.

In one particularly advantageous embodiment of the invention, the pharmaceutical composition comprises the compound of formula (VI) as previously defined, as single active ingredient

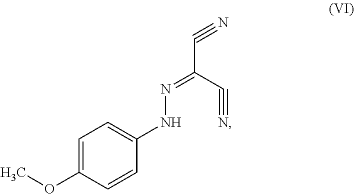

or one of its acceptable pharmaceutical salts and at least one pharmaceutically acceptable excipient and/or a support or a diluent or a pharmaceutically acceptable carrier.

In one particularly advantageous embodiment of the invention, the pharmaceutical composition comprises the compound of formula (VII) as previously defined, as single active ingredient

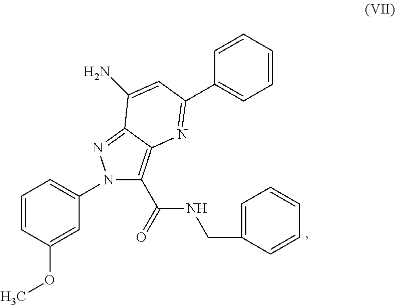 [SMILES: COc1cccc(-n2nc3c(N)cc(-c4ccccc4)nc3c2C(=O)NCc2ccccc2)c1]

or one of its acceptable pharmaceutical salts and at least one pharmaceutically acceptable excipient and/or a support or a diluent or a pharmaceutically acceptable carrier.

In one particularly advantageous embodiment of the invention, the pharmaceutical composition comprises the compound of formula (VIII) as previously defined, as single active ingredient

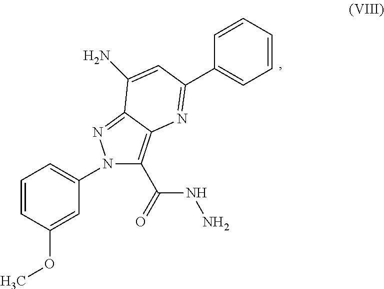 [SMILES: COc1cccc(-n2nc3c(N)cc(-c4ccccc4)nc3c2C(=O)NN)c1]

or one of its acceptable pharmaceutical salts and at least one pharmaceutically acceptable excipient and/or a support or a diluent or a pharmaceutically acceptable carrier.

In one particularly advantageous embodiment of the invention, the pharmaceutical composition comprises the compound of formula (IX) as previously defined, as single active ingredient

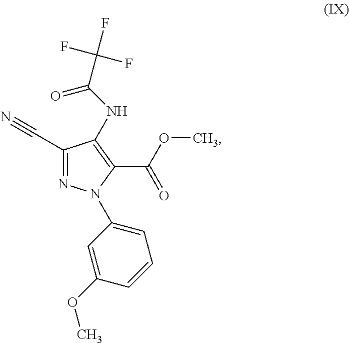 [SMILES: COC(=O)c1c(NC(=O)C(F)(F)F)c(C#N)nn1-c1cccc(OC)c1]

or one of its acceptable pharmaceutical salts and at least one pharmaceutically acceptable excipient and/or a support or a diluent or a pharmaceutically acceptable carrier.

In one advantageous embodiment, the pharmaceutical composition according to the invention further comprises at least one second active ingredient. In one particularly advantageous embodiment of the invention, the at least one second active ingredient can interact synergistically with the compound of formula (I) according to the invention or a pharmaceutically acceptable salt thereof. Advantageously, the second active ingredient can be a corticosteroid, advantageously prednisone.

In one advantageous embodiment of the invention, when the pharmaceutical composition comprises a second active ingredient, the pharmaceutical composition is suitable for simultaneous administration or sequential administration of the compound of formula (I) according to the invention or a pharmaceutically acceptable salt thereof and of the second active ingredient.

Within the meaning of the present invention, “simultaneous administration” is understood to mean administration of the compound of formula (I) according to the invention or a pharmaceutically acceptable salt thereof and of the second active ingredient at the same time, at the same instant, to a patient in therapeutically effective amounts to allow the synergistic effect of the pharmaceutical composition. This does not mean that the compound of formula (I) according to the invention or a pharmaceutically acceptable salt thereof and the second active ingredient are necessarily administered in the form of a mixture; they may indeed be administered simultaneously but separately, in the form of discrete compositions.

“Present in two discrete compositions” is understood to mean that the compound of formula (I) according to the invention or a pharmaceutically acceptable salt thereof and the second active ingredient are physically separate. They are then implemented, administered separately in therapeutically effective amounts to allow the synergistic effect of the pharmaceutical composition, without prior mixing, in several (at least two) dosage forms (for example, two discrete compositions).

In another particular embodiment of the invention, the compound of formula (I) according to the invention or a pharmaceutically acceptable salt thereof and the second active ingredient may be present in a single composition in therapeutically effective amounts to allow the synergistic effect of the pharmaceutical composition. Within the meaning of the present invention, “present in a single composition” is understood to mean the physical combination of the compound of formula (I) according to the invention or a pharmaceutically acceptable salt thereof and the second active ingredient. The compound of formula (I) according to the invention or a pharmaceutically acceptable salt thereof and the second active ingredient are then necessarily administered simultaneously since they are administered together, in the form of a mixture, in a single dosage form (for example, in a single composition containing the compound of formula (I) according to the invention and prednisone) and in therapeutically effective amounts to allow the synergistic effect of the pharmaceutical composition.

Within the meaning of the present invention, the expression “sequential administration” means that the compound of formula (I) according to the invention or a pharmaceutically acceptable salt thereof and the second active ingredient are administered in therapeutically effective amounts to allow the synergistic effect of the pharmaceutical composition, not simultaneously but separately over time, one after the other. The terms “precede” or “preceding” and “follow” or “following” then apply. The term “precede” or “preceding” is used when one compound of the pharmaceutical composition according to the invention is administered a few minutes or several hours, or even several days before the administration of the other compound(s) of the pharmaceutical composition. Conversely, the term “follow” or “following” is used when one compound of the pharmaceutical composition is administered a few minutes or several hours, or even several days after the administration of the other compound(s) of the pharmaceutical composition. In one particularly advantageous embodiment of the invention, the compound of formula (I) according to the invention or a pharmaceutically acceptable salt thereof is administered a few days before the second active ingredient.

In one advantageous embodiment of the invention, when the pharmaceutical composition comprises a second active ingredient, the pharmaceutical composition is suitable for sequential administration of the compound of formula (I) according to the invention or a pharmaceutically acceptable salt thereof and of the second active ingredient.

Advantageously, the second active ingredient may be a corticosteroid, advantageously prednisone.

In one particular embodiment of the invention, the compound of formula (I) according to the invention or a pharmaceutically acceptable salt thereof is administered to the patient, then two to three days after, the second active ingredient is administered to this same patient, for at least one week, advantageously at least two weeks, advantageously at least three weeks, advantageously at least four weeks, advantageously at least five weeks, advantageously at least six weeks, advantageously at least seven weeks.

Administration during in vivo treatment can be done by any route, including oral, parenteral, transdermal, intramuscular, intranasal, sublingual, intratracheal, by inhalation, ocular, vaginal and rectal. Intracapsular, intravenous and intraperitoneal administration routes can also be used. A skilled person will know how to adapt the administration route based on the disorder to be treated. For example, the compound of formula (I) according to the invention or a pharmaceutically acceptable salt thereof or the pharmaceutical composition according to the invention comprising at least one compound of formula (I) according to the invention or a pharmaceutically acceptable salt thereof, can be administered to a patient by oral, parenteral or topical administration. In one embodiment, the compound of formula (I) according to the invention or a pharmaceutically acceptable salt thereof or the pharmaceutical composition according to the invention comprising at least one compound of formula (I) according to the invention or a pharmaceutically acceptable salt thereof is in a form suitable for its administration by oral, parenteral or topical route. In one embodiment, the compound of formula (I) according to the invention or a pharmaceutically acceptable salt thereof or the pharmaceutical composition according to the invention comprising at least one compound of formula (I) according to the invention or a pharmaceutically acceptable salt thereof, optionally in the form of nanoparticles, are administered by intravenous infusion.

The compound of formula (I) according to the invention or a pharmaceutically acceptable salt thereof or the pharmaceutical composition according to the invention comprising at least one compound of formula (I) according to the invention or a pharmaceutically acceptable salt thereof, when it is desirable to administer them systemically, can be formulated for parenteral administration by injection, for example, by bolus injection or continuous infusion. The formulations for injection may be presented in a unit dosage form, for example in ampoules or in multi-dose containers, with an added preservative. The pharmaceutical compositions may take forms such as suspensions, solutions or emulsions in oily or aqueous carriers and may contain formulation agents such as suspending agents, stabilizers and/or dispersants.

Pharmaceutical formulations for parenteral administration comprise aqueous solutions of active pharmaceutical compositions in water-soluble form. In addition, suspensions of the active pharmaceutical compositions may be prepared in the form of suitable oily suspensions for injection. Suitable lipophilic solvents or carriers comprise fatty oils such as sesame oil, or synthetic fatty acid esters, such as ethyl oleate or triglycerides, or liposomes.

Aqueous injection suspensions may contain substances that increase the viscosity of the suspension, such as sodium carboxymethylcellulose, sorbitol or dextran.

Optionally, the suspension may also contain suitable stabilizers or agents which increase the solubility of the pharmaceutical compositions to allow the preparation of highly concentrated solutions. Alternatively, the active compositions may be in powder form to constitute a suitable carrier, for example, pyrogen-free sterile water, before use.

For oral administration, the pharmaceutical compositions may take the form of, for example, tablets or capsules prepared by conventional means with pharmaceutically acceptable excipients such as binding agents (for example, pregelatinized corn starch, polyvinylpyrrolidone, methylcellulose or hydroxypropyl methylcellulose); fillers (for example, lactose, microcrystalline cellulose or calcium hydrogen phosphate); lubricants (for example, magnesium stearate, talc or silica); disintegrating agents (for example, potato starch or sodium starch glycolate); or wetting agents (for example, sodium lauryl sulfate). The tablets can be coated by processes that are well known in the art. Liquid preparations for oral administration may take the form of, for example, solutions, syrups or suspensions, or may be presented as a dry product for reconstitution with water or another suitable carrier before use.

These liquid preparations can be prepared by conventional means with pharmaceutically acceptable additives such as suspending agents (for example, sorbitol syrup, cellulose derivatives or hydrogenated edible fats); emulsifiers (for example, lecithin or gum arabic); non-aqueous carriers (for example, almond oil, oily esters, ethyl alcohol or fractionated vegetable oils); and preservatives (for example, methyl or propyl-p-hydroxybenzoates or sorbic acid). The preparations may also contain buffer salts, flavorings, colorants and sweeteners, depending on the case. It is also desired to increase the overall stability of the compounds of formula (I) as defined and to increase the circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Other polymers that can be used are poly-1,3-dioxolane and poly-1,3,6-tioxocane. As noted above, polyethylene glycol molecules are preferred for pharmaceutical use. For oral compositions, the site of release can be the stomach, the small intestine (duodenum, jejunum or ileum) or the large intestine.

A skilled person knows formulations that do not dissolve in the stomach, but which will release the material into the duodenum or elsewhere in the intestine. Preferably, the release avoids the deleterious effects of the environment of the stomach, by releasing the biologically active material beyond the environment of the stomach, such as into the intestine.

For administration by mouth, the compositions may take the form of conventionally formulated tablets or lozenges.

For administration by inhalation, the compositions to be used according to the present invention may be suitably delivered in the form of an aerosol spray from pressurized packages or a nebulizer, using a suitable propellant, for example, dichlorodifluoromethane, trichlorofluoromethane, dichlorotetrafluoroethane, carbon dioxide or another suitable gas. In the case of a pressurized aerosol, the dosage unit can be determined by providing a valve to deliver a measured amount. The compositions according to the invention may be reduced to powder form for administration by inhalation. In this case, the compositions according to the invention in powder form may be mixed with a second powder, referred to as base powder, such as a lactose or starch powder, the mixture of the two powders being packaged in the form of cartridges or capsules intended to be used in an inhaler or an insufflator.

The present invention also relates to pulmonary administration. The pharmaceutical compositions can be delivered to the lungs of a mammal while inhaling and cross the epithelial mucosa of the lung into the bloodstream. A wide range of mechanical devices designed for the pulmonary delivery of therapeutic products, comprising, but not limited to, nebulizers, metered-dose inhalers and powder inhalers, all known to those skilled in the art, are contemplated for use in the present invention.

Nasal delivery of a pharmaceutical composition described herein is also contemplated. Nasal delivery allows the passage of a pharmaceutical composition of the present invention into the bloodstream directly after the administration of the therapeutic product to the nose, without the need to deposit the product in the lungs. Formulations for nasal administration comprise those with dextran or cyclodextrin, as well as bioadhesive excipients such as, for example, chitosan.

The pharmaceutical compositions can also be formulated in rectal or vaginal compositions such as suppositories or retention enemas, for example containing conventional suppository bases such as cocoa butter or other glycerides.

The pharmaceutical compositions may also comprise suitable solid or gel phase supports or excipients. Examples of such supports or excipients comprise, but are not limited to, calcium carbonate, calcium phosphate, various sugars, starches, cellulose derivatives, gelatin and polymers such as polyethylene glycols.

Suitable liquid or solid pharmaceutical preparation forms are, for example, aqueous or saline solutions for inhalation, which are microencapsulated, notched, applied to microscopic gold particles contained in liposomes, nebulized, aerosols, pastilles for implantation in the skin. The pharmaceutical compositions also comprise granules, powders, tablets, coated tablets, (micro)capsules, suppositories, syrups, emulsions, suspensions, creams, cutaneous or transdermal patches, drops or sustained release preparations of active compositions and/or auxiliaries such as disintegrants, binding agents, coating agents, bulking agents, lubricants, flavorings, sweeteners or solubilizers are usually used as described hereinbefore. The pharmaceutical compositions are suitable for use in various drug administration systems.

Another subject matter of the invention relates to a method for preventive treatment of inflammatory diseases, comprising the administration to the patient of a compound of formula (I) according to the invention or a pharmaceutically acceptable salt thereof or the pharmaceutical composition according to the invention comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

Within the meaning of the present invention, the term “prevention” or “prophylaxis” or “preventive treatment” or “prophylactic treatment” comprises a treatment leading to the prevention of a disease and a treatment reducing and/or delaying the incidence of a disease or the risk of the disease occurring. In one particular embodiment of the invention, the present invention relates to a method for preventive treatment of inflammatory diseases, comprising the administration to the patient of a compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined and a second active ingredient as previously defined.

Advantageously, the inflammatory disease may be selected from: amyloid neurodegenerative diseases, such as Alzheimer's disease, prions, Parkinson's disease, amyotrophic lateral sclerosis, inflammatory bowel diseases, such as chronic inflammatory bowel disease (CIBD), irritable bowel syndrome (IBS), Crohn's disease, ulcerative colitis, hemorrhagic rectal ulcer, concomitant lesions of Behçet's disease, pouchitis, ulcerative colitis, ileitis and enteritis; acute inflammatory diseases such as gout and septic shock, chronic inflammatory low back pain, inflammatory skin diseases or dermatitis, such as psoriasis, atopic dermatitis, rosacea, couperosis, acne, warts, bullous skin diseases, contact dermatitis, skin cancers, redness, erythema, telangiectasia, skin inflammations linked to UV exposure, such as photo-irritation, photo-sensitization, photoaging, photo-carcinogenesis, lymphatic venous insufficiency or heavy legs syndrome; arthritis, such as osteoarthritis, rheumatoid arthritis, juvenile idiopathic arthritis and psoriatic arthritis; chronic obstructive pulmonary disease (COPD); celiac disease; chronic pancreatitis; Hashimoto's thyroiditis; primary biliary cirrhosis; sclerosing cholangitis; autoimmune hepatitis; vasculitides such as systemic vasculitides associated with ANCA (anti-neutrophil cytoplasmic antibodies); spondyloarthropathies; chronic atrophic polychondritis; diabetes; scleroderma and cancer, the list being non-exhaustive.

In one particularly advantageous embodiment, the present invention relates to a method for preventive treatment of amyloid neurodegenerative diseases, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

Advantageously, the amyloid neurodegenerative diseases can be selected from: Alzheimer's disease, prions, Parkinson's disease, amyotrophic lateral sclerosis, the list being non-exhaustive.

In one particularly advantageous embodiment, the present invention relates to a method for preventive treatment of acute inflammatory diseases, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

Advantageously, the acute inflammatory diseases can be selected from: gout and septic shock, the list being non-exhaustive.

In one particularly advantageous embodiment, the present invention relates to the use of a compound of formula (I) according to the invention or a pharmaceutically acceptable salt thereof as an active ingredient in the manufacture of a drug intended for the prevention of inflammatory diseases.

In one particularly advantageous embodiment, the present invention relates to the use of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined in the manufacture of a drug intended for the prevention of inflammatory diseases.

In one particularly advantageous embodiment, the present invention relates to a compound of formula (I) according to the invention or a pharmaceutically acceptable salt thereof for its use in the prevention of inflammatory diseases. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the prevention of inflammatory diseases.

In one particularly advantageous embodiment, the present invention relates to a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined for its use in the prevention of inflammatory diseases. Advantageously, the present invention relates to a pharmaceutical composition comprising a therapeutically effective amount of at least one compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the prevention of inflammatory diseases.

Another subject matter of the invention relates to a method for preventive treatment of autoimmune diseases, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

According to the present invention, the compound of formula (I) according to the invention or a pharmaceutically acceptable salt thereof is particularly effective in preventing the appearance of autoimmune diseases, by inhibiting the production of inflammatory cytokines, thus inducing a protective effect against autoimmune diseases.

In one particular embodiment of the invention, the present invention relates to a method for preventive treatment of autoimmune diseases, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined and a second active ingredient as previously defined.

Advantageously, the autoimmune disease may be selected from: disseminated lupus erythematosus, systemic lupus erythematosus, multiple sclerosis, rheumatoid polyarthritis, insulin-dependent diabetes, idiopathic thrombocytopenic purpura (ITP), transplant or organ rejection, graft-versus-host disease, the various types of sclerosis, primary Sjögren's syndrome (or Gougerot-Sjögren syndrome), autoimmune polyneuropathies such as multiple sclerosis, type I diabetes, autoimmune hepatitis, ankylosing spondylitis, Reiter's syndrome, gouty arthritis, chronic Hashimoto's thyroiditis (hypothyroidism), Addison's disease, autoimmune hepatitis, Basedow's disease (hyperthyroidism), autoimmune cytopenias and other hematological complications in adults and children, such as acute or chronic autoimmune thrombocytopenia, autoimmune hemolytic anemia, hemolytic disease of the newborn (HDN), cold agglutinin disease, idiopathic thrombocytopenic purpura and autoimmune acquired hemophilia, Goodpasture's syndrome, extra-membranous nephropathy, autoimmune bullous skin disorders, refractory myasthenia gravis, mixed cryoglobulinemia, inflammatory myositis, dermatomyositis and autoimmune systemic disorders in children, including antiphospholipid syndrome and connective tissue disease, the various types of sclerosis, pulmonary autoimmune inflammation, Guillain-Barre syndrome, chronic inflammatory demyelinating polyradiculoneuropathy (CIDP), autoimmune thyroiditis, mellitis, myasthenia gravis, autoimmune inflammatory disease of the eye, neuromyelitis optica (Devic's disease), scleroderma, pemphigus, insulin-resistant diabetes, polymyositis, Biermer's anemia, glomerulonephritis, Wegener's disease, Horton's disease, periarteritis nodosa and Churg-Strauss syndrome, Still's disease, atrophic polychondritis, Behçet's disease, monoclonal gammopathy, Wegener's granulomatosis, lupus, psoriatic arthritis, sarcoidosis, collagenous colitis, dermatitis herpetiformis, familial Mediterranean fever, IgA glomerulonephritis, Lambert-Eaton myasthenic syndrome, sympathetic ophthalmia, Fiessinger-Leroy-Reiter syndrome and uveo-meningo-encephalic syndrome, the list being non-exhaustive.

In one particularly advantageous embodiment, the present invention relates to a method for preventive treatment of rheumatoid polyarthritis, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to a method of preventive treatment of disseminated lupus erythematosus, comprising the administration to the patient of the compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to the use of a compound of formula (I) according to the invention or a pharmaceutically acceptable salt thereof as an active ingredient in the manufacture of a drug intended for the prevention of autoimmune diseases.

In one particularly advantageous embodiment, the present invention relates to the use of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined in the manufacture of a drug intended for the prevention of autoimmune diseases.

In one particularly advantageous embodiment, the present invention relates to a compound of formula (I) according to the invention or a pharmaceutically acceptable salt thereof as previously defined for its use in the prevention of autoimmune diseases. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the prevention of autoimmune diseases.

In one particularly advantageous embodiment, the present invention relates to a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined for its use in the prevention of autoimmune diseases. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the prevention of autoimmune diseases.

Another subject matter of the invention relates to a method for preventive treatment of viral and/or bacterial infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

According to the present invention, the compounds of formula (I) according to the invention or a pharmaceutically acceptable salt thereof are particularly effective in preventing the appearance, development, amplification, persistence of viral and/or bacterial infections, by inhibiting the production of inflammatory cytokines, thus inducing a protective effect against viral and/or bacterial infections. Advantageously, the inventors have also shown that the compounds of formula (V), (VI), (VIII) and (IX) are capable of blocking the production and/or secretion of interferons on peripheral blood mononuclear cells (PBMCs) from healthy donors preventively before stimulation by the influenza virus (flu virus). The inventors have also shown that the compounds of formula (V) and (VI) are capable of blocking the production of TNFα by plasmacytoid dendritic cells isolated from peripheral blood mononuclear cells (PBMCs) from healthy donors preventively before stimulation by the influenza virus (flu virus).

The inventors have also shown that the compounds of formula (V), (VI) and (VIII) are capable of blocking the production and/or secretion of interferons on peripheral blood mononuclear cells (PBMCs) from healthy donors preventively before stimulation by the human immunodeficiency virus (HIV).

Thus, the inventors have shown quite surprisingly that the use of a compound of formula (I) has a preventive effect on the inhibition of interferon production induced by the influenza virus (flu virus) and the human immunodeficiency virus (HIV).

In one particular embodiment of the invention, the present invention relates to a method for preventive treatment of viral and/or bacterial infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined and a second active ingredient as previously defined.

Advantageously, the viral infections can be selected from: infections due to the influenza virus, the human immunodeficiency virus (HIV), the herpes virus or Herpes Simplex Virus (HSV), coronavirus, COVID-19, the encephalomyocarditis virus, arboviruses, such as, Chikungunya, O'Nyong Nyong, Ross River, Sindbis, Mayaro, yellow fever virus, dengue fever, Japanese encephalitis, West Nile virus, temperate Eurasian tick-borne encephalitis viruses, Kyasanur forest disease viruses, Omsk hemorrhagic fever, Bunyaviridae, Bunyamwera virus, Rift Valley fever virus, Crimean Congo hemorrhagic fever virus, hepatitis A, B and C virus and influenza virus, the list being non-exhaustive.

Advantageously, the bacterial infections can be selected from: infections due to gram-positive bacteria, such as bacteria of the Staphylococcus genus, in particular Staphylococcus aureus, and bacteria of the Enterococcus genus, in particular Enterococcus faecalis, infections due to gram-negative bacteria, such as bacteria of the Escherichia genus, in particular Escherichia coli, bacteria of the Pseudomonas genus, in particular Pseudomonas aeruginosa, and bacteria of the Acinetobacter genus, in particular Acinetobacter baumannii, the list being non-exhaustive.

In one particularly advantageous embodiment, the present invention relates to a method for preventive treatment of coronavirus infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to a method for preventive treatment of infections due to the influenza virus, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined

In one particularly advantageous embodiment, the present invention relates to a method for preventive treatment of human immunodeficiency virus (HIV) infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to the use of a compound of formula (I) according to the invention or a pharmaceutically acceptable salt thereof as an active ingredient in the manufacture of a drug intended for the prevention of viral and/or bacterial infections.

In one particularly advantageous embodiment, the present invention relates to the use of a pharmaceutical composition comprising a therapeutically effective amount of a compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined in the manufacture of a drug intended for the prevention of viral and/or bacterial infections.

In one particularly advantageous embodiment, the present invention relates to a compound of formula (I) according to the invention or a pharmaceutically acceptable salt thereof as previously defined for its use in the prevention of viral and/or bacterial infections. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the prevention of viral and/or bacterial infections.

In one particularly advantageous embodiment, the present invention relates to a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined for its use in the prevention of viral and/or bacterial infections. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the prevention of viral and/or bacterial infections.

Another subject matter of the invention relates to a method for curative treatment of inflammatory diseases, comprising the administration to the patient of a compound of formula (I) according to the invention or a pharmaceutically acceptable salt thereof or the pharmaceutical composition according to the invention comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

Within the meaning of the present invention, the term “treatment” or “curative treatment” is defined as a treatment resulting in a healing or a treatment which alleviates, improves and/or eliminates, reduces and/or stabilizes the symptoms of a disease or the suffering that it causes.

In one particular embodiment of the invention, the present invention relates to a method for curative treatment of inflammatory diseases, comprising the administration to the patient of a compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined and a second active ingredient as previously defined.

Advantageously, the inflammatory disease may be selected from: amyloid neurodegenerative diseases, such as Alzheimer's disease, prions, Parkinson's disease, amyotrophic lateral sclerosis, inflammatory bowel diseases, such as chronic inflammatory bowel disease (CIBD), irritable bowel syndrome (IBS), Crohn's disease, ulcerative colitis, hemorrhagic rectal ulcer, concomitant lesions of Behçet's disease, pouchitis, ulcerative colitis, ileitis and enteritis; acute inflammatory diseases such as gout and septic shock, chronic inflammatory low back pain, inflammatory skin diseases or dermatitis, such as psoriasis, atopic dermatitis, rosacea, couperosis, acne, warts, bullous skin diseases, contact dermatitis, skin cancers, redness, erythema, telangiectasia, skin inflammations linked to UV exposure, such as photo-irritation, photo-sensitization, photoaging, photo-carcinogenesis, lymphatic venous insufficiency or heavy legs syndrome; arthritis, such as osteoarthritis, rheumatoid arthritis, juvenile idiopathic arthritis and psoriatic arthritis; chronic obstructive pulmonary disease (COPD); celiac disease; chronic pancreatitis; Hashimoto's thyroiditis; primary biliary cirrhosis; sclerosing cholangitis; autoimmune hepatitis; vasculitides such as systemic vasculitides associated with ANCA (anti-neutrophil cytoplasmic antibodies); spondyloarthropathies; chronic atrophic polychondritis; diabetes; scleroderma and cancer, the list being non-exhaustive.

In one particularly advantageous embodiment, the present invention relates to a method for curative treatment of amyloid neurodegenerative diseases, comprising the administration to the patient of the compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

Advantageously, the amyloid neurodegenerative diseases can be selected from: Alzheimer's disease, prions, Parkinson's disease, amyotrophic lateral sclerosis, the list being non-exhaustive.

In one particularly advantageous embodiment, the present invention relates to a method for curative treatment of acute inflammatory diseases, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

Advantageously, the acute inflammatory diseases can be selected from: gout and septic shock, the list being non-exhaustive.

In one particularly advantageous embodiment, the present invention relates to the use of a compound of formula (I) according to the invention or a pharmaceutically acceptable salt thereof as an active ingredient in the manufacture of a drug intended for the treatment of inflammatory diseases.

In one particularly advantageous embodiment, the present invention relates to the use of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined in the manufacture of a drug intended for the treatment of inflammatory diseases.

In one particularly advantageous embodiment, the present invention relates to a compound of formula (I) according to the invention or a pharmaceutically acceptable salt thereof for its use in the treatment of inflammatory diseases.

Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the treatment of inflammatory diseases.

In one particularly advantageous embodiment, the present invention relates to a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined for its use in the treatment of inflammatory diseases. Advantageously, the present invention relates to a pharmaceutical composition comprising a therapeutically effective amount of at least one compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the treatment of inflammatory diseases.

Another subject matter of the invention relates to a method for curative treatment of autoimmune diseases, comprising the administration to the patient of the compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

According to the present invention, the compound of formula (I) according to the invention or a pharmaceutically acceptable salt thereof is particularly effective in preventing the appearance of autoimmune diseases, by inhibiting the production of inflammatory cytokines, thus inducing a protective effect against autoimmune diseases.

In one particular embodiment of the invention, the present invention relates to a method for curative treatment of autoimmune diseases, comprising the administration to the patient of the compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined and a second active ingredient as previously defined.

Advantageously, the autoimmune disease may be selected from: disseminated lupus erythematosus, systemic lupus erythematosus, multiple sclerosis, rheumatoid polyarthritis, insulin-dependent diabetes, idiopathic thrombocytopenic purpura (ITP), transplant or organ rejection, graft-versus-host disease, the various types of sclerosis, primary Sjögren's syndrome (or Gougerot-Sjögren syndrome), autoimmune polyneuropathies such as multiple sclerosis, type I diabetes, autoimmune hepatitis, ankylosing spondylitis, Reiter's syndrome, gouty arthritis, chronic Hashimoto's thyroiditis (hypothyroidism), Addison's disease, autoimmune hepatitis, Basedow's disease (hyperthyroidism), autoimmune cytopenias and other hematological complications in adults and children, such as acute or chronic autoimmune thrombocytopenia, autoimmune hemolytic anemia, hemolytic disease of the newborn (HDN), cold agglutinin disease, idiopathic thrombocytopenic purpura and autoimmune acquired hemophilia, Goodpasture's syndrome, extra-membranous nephropathy, autoimmune bullous skin disorders, refractory myasthenia gravis, mixed cryoglobulinemia, inflammatory myositis, dermatomyositis and autoimmune systemic disorders in children, including antiphospholipid syndrome and connective tissue disease, the various types of sclerosis, pulmonary autoimmune inflammation, Guillain-Barre syndrome, chronic inflammatory demyelinating polyradiculoneuropathy (CIDP), autoimmune thyroiditis, mellitis, myasthenia gravis, autoimmune inflammatory disease of the eye, neuromyelitis optica (Devic's disease), scleroderma, pemphigus, insulin-resistant diabetes, polymyositis, Biermer's anemia, glomerulonephritis, Wegener's disease, Horton's disease, periarteritis nodosa and Churg-Strauss syndrome, Still's disease, atrophic polychondritis, Behçet's disease, monoclonal gammopathy, Wegener's granulomatosis, lupus, psoriatic arthritis, sarcoidosis, collagenous colitis, dermatitis herpetiformis, familial Mediterranean fever, IgA glomerulonephritis, Lambert-Eaton myasthenic syndrome, sympathetic ophthalmia, Fiessinger-Leroy-Reiter syndrome and uveo-meningo-encephalic syndrome, the list being non-exhaustive.

In one particularly advantageous embodiment, the present invention relates to a method for curative treatment of rheumatoid polyarthritis, comprising the administration to the patient of the compound of formula (I) according to the invention or a pharmaceutically acceptable salt thereof or a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to a method for curative treatment of disseminated lupus erythematosus, comprising the administration to the patient of the compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to the use of a compound of formula (I) according to the invention or a pharmaceutically acceptable salt thereof as an active ingredient in the manufacture of a drug intended for the treatment of autoimmune diseases.

In one particularly advantageous embodiment, the present invention relates to the use of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined in the manufacture of a drug intended for the treatment of autoimmune diseases.

In one particularly advantageous embodiment, the present invention relates to a compound of formula (I) according to the invention or a pharmaceutically acceptable salt thereof as previously defined for its use in the treatment of autoimmune diseases. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the treatment of autoimmune diseases.

In one particularly advantageous embodiment, the present invention relates to a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined for its use in the treatment of autoimmune diseases. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the treatment of autoimmune diseases.

Another subject matter of the invention relates to a method for curative treatment of viral and/or bacterial infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

According to the present invention, the compounds of formula (I) according to the invention or a pharmaceutically acceptable salt thereof are particularly effective in preventing the appearance, development, amplification, persistence of viral and/or bacterial infections, by inhibiting the production of inflammatory cytokines, thus inducing a protective effect against viral and/or bacterial infections. Advantageously, the inventors have also shown that the compounds of formula (V), (VI), (VIII) and (IX) are capable of blocking the production of interferons on peripheral blood mononuclear cells (PBMCs) from healthy donors preventively before stimulation by the influenza virus (flu virus).

The inventors have also shown that the compounds of formula (V) and (VI) are capable of blocking the production of TNFα by plasmacytoid dendritic cells isolated from peripheral blood mononuclear cells (PBMCs) from healthy donors preventively before stimulation by the influenza virus (flu virus).

The inventors have also shown that the compounds of formula (V), (VI) and (VIII) are capable of blocking the production and/or secretion of interferons on peripheral blood mononuclear cells (PBMCs) from healthy donors preventively before stimulation by the human immunodeficiency virus (HIV).

Thus, the inventors have shown quite surprisingly that the use of a compound of formula (I) has a curative effect on the inhibition of interferon production induced by the influenza virus (flu virus) and the human immunodeficiency virus (HIV).

In one particular embodiment of the invention, the present invention relates to a method for curative treatment of viral and/or bacterial infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined and a second active ingredient as previously defined.

Advantageously, the viral infections can be selected from: infections due to the influenza virus, the human immunodeficiency virus (HIV), the herpes virus or Herpes Simplex Virus (HSV), coronavirus, COVID-19, the encephalomyocarditis virus, arboviruses, such as, Chikungunya, O'Nyong Nyong, Ross River, Sindbis, Mayaro, yellow fever virus, dengue fever, Japanese encephalitis, West Nile virus, temperate Eurasian tick-borne encephalitis viruses, Kyasanur forest disease viruses, Omsk hemorrhagic fever, Bunyaviridae, Bunyamwera virus, Rift Valley fever virus, Crimean Congo hemorrhagic fever virus, hepatitis A, B and C virus and influenza virus, the list being non-exhaustive.

Advantageously, the bacterial infections can be selected from: infections due to gram-positive bacteria, such as bacteria of the Staphylococcus genus, in particular Staphylococcus aureus, and bacteria of the Enterococcus genus, in particular Enterococcus faecalis, infections due to gram-negative bacteria, such as bacteria of the Escherichia genus, in particular Escherichia coli, bacteria of the Pseudomonas genus, in particular Pseudomonas aeruginosa, and bacteria of the Acinetobacter genus, in particular Acinetobacter baumannii, the list being non-exhaustive.

In one particularly advantageous embodiment, the present invention relates to a method for curative treatment of coronavirus infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to a method for curative treatment of infections due to the influenza virus, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined

In one particularly advantageous embodiment, the present invention relates to a method for curative treatment of human immunodeficiency virus (HIV) infections, comprising the administration to the patient of a compound of formula (I) according to the invention or a pharmaceutically acceptable salt thereof or of a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined.

In one particularly advantageous embodiment, the present invention relates to the use of a compound of formula (I) according to the invention or a pharmaceutically acceptable salt thereof as an active ingredient in the manufacture of a drug intended for the treatment of viral and/or bacterial infections.

In one particularly advantageous embodiment, the present invention relates to the use of a pharmaceutical composition comprising a therapeutically effective amount of a compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined in the manufacture of a drug intended for the treatment of viral and/or bacterial infections.

In one particularly advantageous embodiment, the present invention relates to a compound of formula (I) according to the invention or a pharmaceutically acceptable salt thereof as previously defined for its use in the treatment of viral and/or bacterial infections. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the treatment of viral and/or bacterial infections.

In one particularly advantageous embodiment, the present invention relates to a pharmaceutical composition comprising a therapeutically effective amount of at least one compound of formula (I) according to the invention or a pharmaceutically acceptable salt thereof as an active ingredient and at least one pharmaceutically acceptable excipient and/or support and/or a diluent and/or a pharmaceutically acceptable carrier as previously defined for its use in the treatment of viral and/or bacterial infections. Advantageously, the present invention relates to a compound of formula (I) according to the invention or a pharmaceutically acceptable salt thereof selected from: the compounds of formula (V), of formula (VI), of formula (VII), of formula (VIII) and of formula (IX) as an active ingredient and at least one pharmaceutically acceptable excipient and/or support and/or a diluent and/or a pharmaceutically acceptable carrier as previously defined for its use in the treatment of viral and/or bacterial infections.

### FIGURES

FIG. 1 shows the modelling of the ROCK and PDK1 kinase complex carried out on the basis of the crystallographic data deposited in the PDB (2ESM for ROCK1 and 2R7B for PDK1). In the ROCK and PDK1 interaction zone, the chemical determinants producing a steric clash between the two kinases and therefore likely to interfere with the assembly of the two enzymes are modeled. The ball structure denotes the side chain of the PDK1 amino acid phosphorylated by ROCK.

FIG. 2 shows the blockage by the compound of formula (V) of the secretion of interferons and inflammatory cytokines on peripheral blood mononuclear cells (PBMCs) from healthy donors, preventively, 1 h before stimulation by toll-like receptors TLR7/8. The results were obtained with the THP1-Dual reporter cell line.

FIG. 3 shows the blockage by the compound of formula (VI) of the secretion of interferons and inflammatory cytokines on peripheral blood mononuclear cells (PBMCs) from healthy donors, preventively, 1 h before stimulation by toll-like receptors TLR7/8. The results were obtained with the THP1-Dual reporter cell line.

FIG. 4 shows the blockage by the compound of formula (VII) of the secretion of interferons of peripheral blood (PBMCs) from healthy donors, preventively, 1 h before stimulation by toll-like receptors TLR7/8. The results were obtained with the STING-37 reporter cell line.

FIG. 5 shows the blockage by the compound of formula (VIII) of the secretion of interferons on peripheral blood mononuclear cells (PBMCs) from healthy donors, preventively, 1 h before stimulation by toll-like receptors TLR7/8. The results were obtained with the STING-37 reporter cell line.

FIG. 6 shows the blockage by the compound of formula (IX) of the secretion of interferons on peripheral blood mononuclear cells (PBMCs) from healthy donors, preventively, 1 h before stimulation by toll-like receptors TLR7/8. The results were obtained with the STING-37 reporter cell line.

FIG. 7 shows the blockage by the compound of formula (V) of the secretion of inflammatory cytokines and interferons on peripheral blood mononuclear cells (PBMCs) from healthy donors in response to stimulation by toll-like receptors TLR7/8. The results were obtained by LegendPlex.

FIG. 8 shows the blockage by the compound of formula (V) of the secretion of inflammatory cytokines and interferons on peripheral blood mononuclear cells (PBMCs) from healthy donors in response to stimulation by toll-like receptors TLR4. The results were obtained by LegendPlex.

FIG. 9 shows the blockage by the compound of formula (VI) of the secretion of inflammatory cytokines and interferons on peripheral blood mononuclear cells (PBMCs) from healthy donors in response to stimulation by toll-like receptors TLR7/8. The results were obtained by LegendPlex.

FIG. 10 shows the blockage by the compound of formula (VI) of the secretion of inflammatory cytokines and interferons on peripheral blood mononuclear cells (PBMCs) from healthy donors in response to stimulation by toll-like receptors TLR4. The results were obtained by LegendPlex.

FIG. 11 shows the blockage by the compounds of formula (V) to (VIII) of the activation of the B lymphocytes of healthy donors in response to stimulation by toll-like receptors TLR9. Depiction of the MFI in a histogram. The results were obtained by flow cytometry.

FIG. 12 shows the blockage by the compounds of formula (V), (VI), (VIII) and (IX) of the secretion of interferons on peripheral blood mononuclear cells (PBMCs) from healthy donors, preventively, 1 h before stimulation by the influenza virus (Flu). The results were obtained with the THP1-Dual reporter cell line.

FIG. 13 shows the blockage by the compounds of formula (V), (VI) and (VIII) of the secretion of interferons on peripheral blood mononuclear cells (PBMCs) from healthy donors, preventively, 1 h before stimulation by the HIV virus. The results were obtained with the THP1-Dual reporter cell line.

FIG. 14 shows the blockage by the compounds of formula (V) and (VI) of the production of TNFα on monocytes isolated from peripheral blood mononuclear cells (PBMCs) from healthy donors, preventively, (A) 1 h before stimulation of TLR7/8, (B) 1 h before stimulation of TLR4, and (C) 1 h before stimulation of TLR2. Depiction of the MFI in a histogram. The results were obtained by flow cytometry.

FIG. 15 shows the blockage by the compounds of formula (V) and (VI) of the production of TNFα on plasmacytoid dendritic cells isolated from peripheral blood mononuclear cells (PBMCs) from healthy donors, preventively, (A) 1 h before stimulation of TLR7, (B) 1 h before stimulation by the influenza virus (Flu), and (C) 1 h before stimulation by TLR9. The results were obtained by flow cytometry.

FIG. 16 shows the blockage by the compound of formula (V), according to a dose effect, of the activation of the NF-κB transcription factor in the THP1-Dual monocytic cell line, in response to the stimulation of toll-like receptors TLR7/8 by R848. The results were obtained by absorptiometry (SEAP assay—gray bars). The toxicity of the compound of formula (V) with respect to the THP1 cells was measured in parallel (WST-1 assay—black bars).

FIG. 17 shows the blockage by the compound of formula (VI), according to a dose effect, of the activation of the NF-κB transcription factor in the THP1-Dual monocytic cell line, in response to the stimulation of toll-like receptors TLR7/8 by R848. Results obtained by absorptiometry (SEAP assay—gray bars). The toxicity of the compound of formula (VI) with respect to the THP1 cells was measured in parallel (WST-1 assay—black bars).

FIG. 18 shows the blockage by the compound of formula (V), according to a dose effect, of the activation of the NF-κB transcription factor in the THP1-Dual monocytic cell line, in response to the stimulation of toll-like receptors TLR4 by LPS. The results were obtained by absorptiometry (SEAP assay—gray bars). The toxicity of the compound of formula (V) with respect to the THP1 cells was measured in parallel (WST-1 assay—black bars).

FIG. 19 shows the blockage by the compound of formula (VI), according to a dose effect, of the activation of the NF-κB transcription factor in the THP1-Dual monocytic cell line, in response to the stimulation of toll-like receptors TLR4 by LPS. The results were obtained by absorptiometry (SEAP assay—gray bars). The toxicity of the compound of formula (VI) with respect to the THP1 cells was measured in parallel (WST-1 assay—black bars).

FIG. 20 shows the blockage by the compounds of formula (V), (VI), (VIII) and (IX) of the activation of the IRF transcription factors in the THP1-Dual monocytic cell line, in response to the stimulation of the STING interferon pathway by cGAMP. The results were obtained by luminometry (gray bars). The toxicity of the compounds of formula (V), (VI), (VIII) and (IX) with respect to the THP1 cells was measured in parallel (WST-1 assay—black bars).

FIG. 21 shows that the compounds of formula (V) and (VI) have no impact on the enzymatic activity of pyruvate kinase (PK).

FIG. 22 shows the blockage by the compound of formula (V) and (VI), according to a dose effect, of the transcription of the pro-inflammatory genes IL-1@, TNFα, IL-6 and MCP1 in the THP1 monocytic cell line, in response to the stimulation of toll-like receptors TLR7/8 by R848. The results were obtained by RT-qPCR.

FIG. 23 shows the blockage by the compounds of formula (V) and (VI), according to a dose effect, of the transcription of the pro-inflammatory genes IL-13 and TNFα in the THP1 monocytic cell line, in response to the stimulation of the toll-like receptors TLR4 by LPS. The results were obtained by RT-qPCR.

FIG. 24 shows the modelling of the ROCK1 kinase in a complex with the compound of formula (V), carried out on the basis of the crystallographic data deposited in the Protein Data Bank (reference 2ESM for ROCK1). The amino acids of ROCK1 involved in the interaction with the compound of formula (V) are indicated.

FIG. 25 shows that the anti-inflammatory properties of the compounds of formula (V) and (VI) are dependent on the PDK1 kinase. The expression of the PDK1 kinase was repressed by a specific siRNA (siPDK1) in monocytes from healthy donors. A control siRNA (siCTL) was used as a negative control for the experiment. The monocytes were then treated with the compounds of formulas (V) or (VI) and stimulated by the TLR7/8 agonist, the compound R848. The intracellular production of TNFα was measured by flow cytometry. (A) Depiction in a dot plot. (B) Depiction of the MFI in a histogram (TNFα).

### EXAMPLES

### Example 1: Effect of the Compounds of Formula (V) to (IX) on the Production of Interferon and the NF-κB Transcription Factor on Human Peripheral Blood Mononuclear Cells

### Preparation of the Compounds of Formula (V), (VI), (VII), (VIII) and (IX)

The compounds of the formulas can be obtained according to the synthesis process described in the publication by Le Corre et al., Microwave-assisted preparation of 4-amino-3-cyano-5-methoxycarbonyl-N-arylpyrazoles as building blocks for the diversity-oriented synthesis of pyrazole-based polycyclic scaffolds, Organic & Biomolecular Chemistry, 2015, vol 13, pages 409-423.

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). The peripheral blood mononuclear cells (PBMCs) were cultured in RPMI 1640 (Sigma) containing 10% inactivated fetal calf serum and 1 mM glutamine (Hyclone, Logan, UT).

### Cell Stimulation

The PBMCs were cultured at 2×10⁶ cells/ml. The cells were then incubated with the compounds of the invention at the indicated concentrations for 1 h before stimulation. The PBMCs were then stimulated for 24 h with the TLR7/8 agonist Resiquimod (R848) at 5 μg/ml. The supernatants were collected for cytokine detection.

### Cytokine Detection

The activity of the NF-κB transcription Factor and the induction of the interferon (IFN) pathway via interferon regulatory factors (IRFs) were measured using the THP1-Dual reporter cell line from InvivoGen. The PBMC culture supernatants were added to the THP1-Dual cultures at a rate of 0.4×10⁶ cells/ml for 24 h. The activity of the NF-κB transcription factor was measured with QUANTI-Blue, a SEAP detection agent following the supplier's recommendations. The activity of the IRFs was measured with QUANTI-Luc, a luciferase detection reagent following the supplier's recommendations.

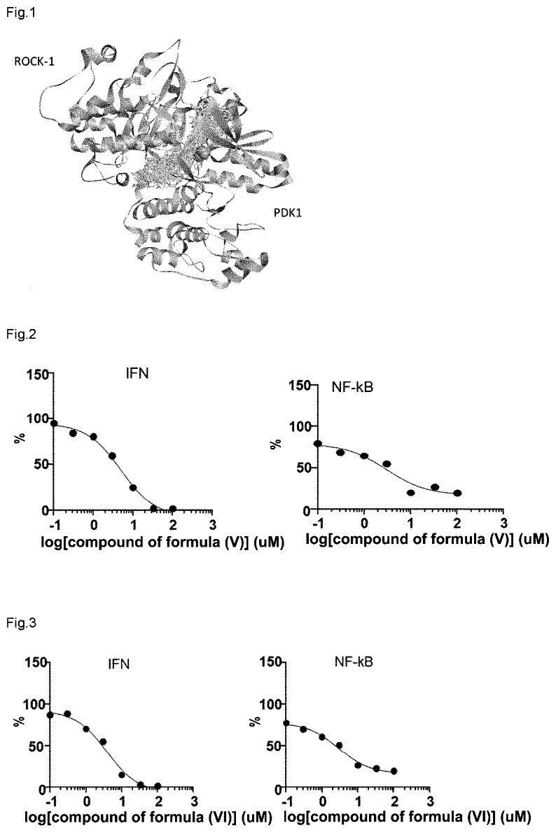

The results are presented in FIGS. 2 to 6.

The IC₅₀ values, determined with respect to the inhibition of the NF-κB transcription factor and of the IRFs, are reproduced in Table 2 hereunder. The interaction scores (virtual score) between the compounds of formulas V to IX and the ROCK1 kinase, determined from the modelling of the ROCK1 kinase with the disruptors of the ROCK/PDK1 complex, are reproduced in Table 2 hereunder.

TABLE 2
IC₅₀ values and interaction scores.

| | | | Virtual score |
|---|---|---|---|
| Compounds | IC₅₀ NF-κB (μM) | IC₅₀ IFR (μM) | (cDocker) |
| Compound of | 3.8 | 3 | 36.8 |
| formula (V) | | | |
| Compound of | 5 | 3 | 38.01 |
| formula (VI) | | | |
| Compound of | Not determined | 23 | 30.86 |
| formula (VII) | | | |
| Compound of | Not determined | 23 | 32.19 |
| formula (VIII) | | | |
| Compound of | Not determined | 0.4 | 53.81 |
| formula (IX) | | | |

### Conclusion

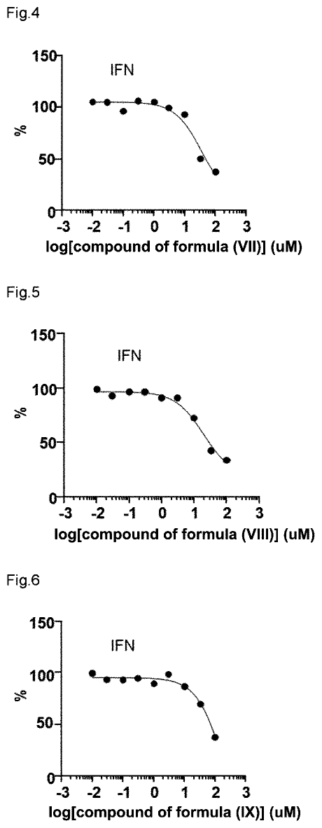

It emerges that:
the compounds of formula (V) and (VI) are capable of blocking the secretion of interferons and inflammatory cytokines on peripheral blood mononuclear cells (PBMCs) from healthy donors, preventively, 1 h before stimulation by toll-like receptors TLR7/8 (FIGS. 2 to 3).
the compounds of formula (VII), (VIII) and (IX) are capable of blocking the secretion of interferons on peripheral blood mononuclear cells (PBMCs) from healthy donors, preventively, 1 h before stimulation by toll-like receptors TLR7/8 (FIGS. 4 to 6).
The compounds of the invention have high affinity for the ROCK1 protein kinase (virtual score higher than 30).

### Example 2: Effect of the Compound of Formula (V) and (VI) on the Production of Interferon and Inflammatory Cytokines on Human Peripheral Blood Mononuclear Cells after Stimulation with R848 and LPS

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). The peripheral blood mononuclear cells (PBMCs) were cultured in RPMI 1640 (Sigma) containing 10% inactivated fetal calf serum and 1 mM glutamine (Hyclone, Logan, UT).

### Cell Stimulation

The PBMCs were cultured at 2×10⁶ cells/ml. The cells were then incubated with the compounds of formula (V) and (VI) at the indicated concentrations for 1 hour before stimulation. The PBMCs were then stimulated for 24 h with the TLR7/8 agonist Resiquimod—R848 at 5 μg/ml and the TLR4 agonist Lipopolysaccharide—LPS at 100 ng/ml. The supernatants were collected for cytokine detection.

### Cytokine Detection

An assay of the cytokines in the supernatants of the cell cultures was carried out using the “LegendPlex Antivirus Human Panel” or “LegendPlex Human Inflammation Panel” (Biolegend, San Diego, USA) according to the manufacturer's instructions.

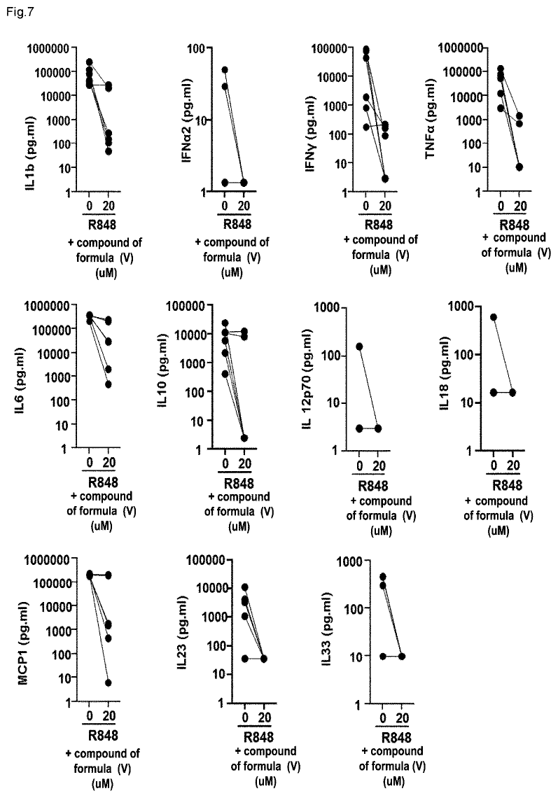

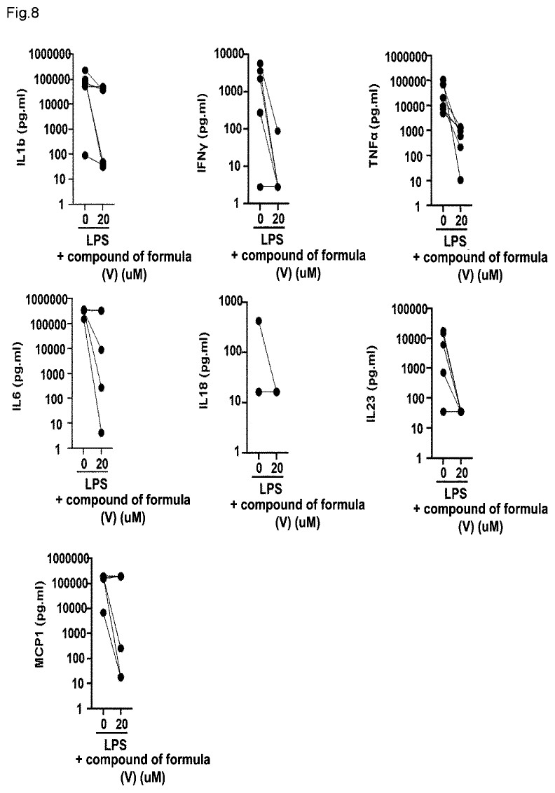

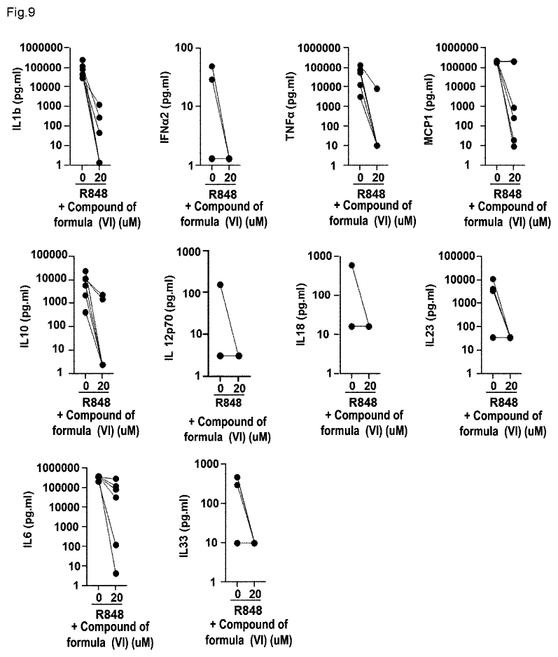

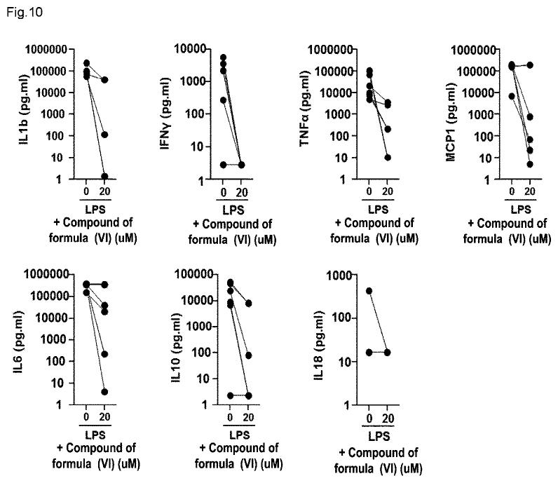

The results are presented in FIGS. 7 to 10.

### Conclusion

It emerges that:
the compounds of formula (V) and (VI) are respectively capable of blocking the secretion of the cytokines TNFα, IL-1β, IL-6, IL-10, IL-12 p70, IL-18, IL-23 and IL-33, as well as the interferons IFNα2 and IFNγ, and the MCP1 protein on peripheral blood mononuclear cells (PBMCs) from healthy donors, in response to stimulation by toll-like receptors TLR7/8.
the compounds of formula (V) and (VI) are respectively capable of blocking the secretion of the cytokines TNFα, IL-1β, IL-6, IL-18 and IL-23, as well as interferon IFNγ, and the MCP1 protein on peripheral blood mononuclear cells (PBMCs) from healthy donors, in response to stimulation by toll-like receptors TLR4.

### Example 3: Effect of the Compounds of Formula (V), (VI), (VII) and (VIII) on the Activation of B Lymphocytes from Healthy Donors in Response to Stimulation by Toll-Like Receptors TLR9

### Isolation and Culture of Blood Leukocytes.

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). The B lymphocytes were purified by negative selection with the “B Cell Isolation Kit” (Miltenyi). The B lymphocytes were cultured in RPMI 1640 (Sigma) containing 10% inactivated fetal calf serum and 1 mM glutamine (Hyclone, Logan, UT).

### Cell Stimulation

The B lymphocytes were cultured at 1×10⁶/ml. The cells were then incubated with the compounds of formula (V) to (VIII), respectively, at 20 μM for 1 h before stimulation. They were stimulated for 16 h (flow cytometry) with the TLR9 agonist CpG-A at 5 mM. The cells were recovered for flow cytometry.

### Flow Cytometry

After washing, the cells were resuspended in PBS containing 2% of FCS and 2 mM of EDTA and then labeled with an anti-CD69 antibody (clone REA824) from Miltenyi Biotec, used at 1/100° for 30 min at 4° C. The cells were washed twice and then passed into flow cytometry. The data acquisition was carried out on the Canto II flow cytometer using Diva software (BD Biosciences, San Jose, CA). FlowJo software was used to analyze the data.

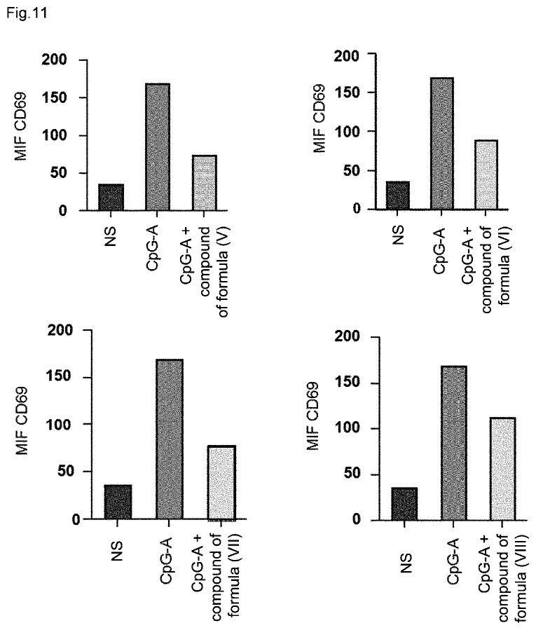

The results are presented in FIG. 11.

### Conclusion

It emerges that all the compounds of formula (V), (VI), (VII) and (VIII) are capable of blocking the activation of the B lymphocytes illustrated by the CD69 membrane activation marker from healthy donors in response to stimulation by toll-like receptors TLR9.

### Example 4: Effect of the Compounds of Formula (V), (VI), (VIII) and (IX) on the Secretion of Interferons by Peripheral Blood Mononuclear Cells (PBMCs) from Healthy Donors, Preventively, 1 h Before Stimulation by the Influenza Virus

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). The peripheral blood mononuclear cells (PBMCs) were cultured in RPMI 1640 (Sigma) containing 10% inactivated fetal calf serum and 1 mM glutamine (Hyclone, Logan, UT).

### Cell Stimulation

The PBMCs were cultured at 2×10⁶ cells/ml. The cells were then incubated with the compounds of formula (V), (VI), (VIII) and (IX) at 20 μM for 1 hour before stimulation. The PBMCs were then stimulated for 24 h with the influenza virus (Flu). The supernatants were collected for cytokine detection.

### Test of Compound Toxicity on the Peripheral Blood Mononuclear Cells (PBMCs)

After 24 h of cell culture, cell viability was determined on the PBMCs using the CellTiter-Glo reagent (Promega), which evaluates the amount of ATP by luminescence using the EnSpire.

### Cytokine Detection

The induction of the interferon (IFN) pathway via the IRFs was measured using the THP1-Dual reporter cell line from InvivoGen. The PBMC culture supernatants were added to THP1-Dual cultures at 0.4×10⁶ cells/ml for 24 h. The activity of the IRFs was measured with QUANTI-Luc, a luciferase detection reagent following the supplier's recommendations.

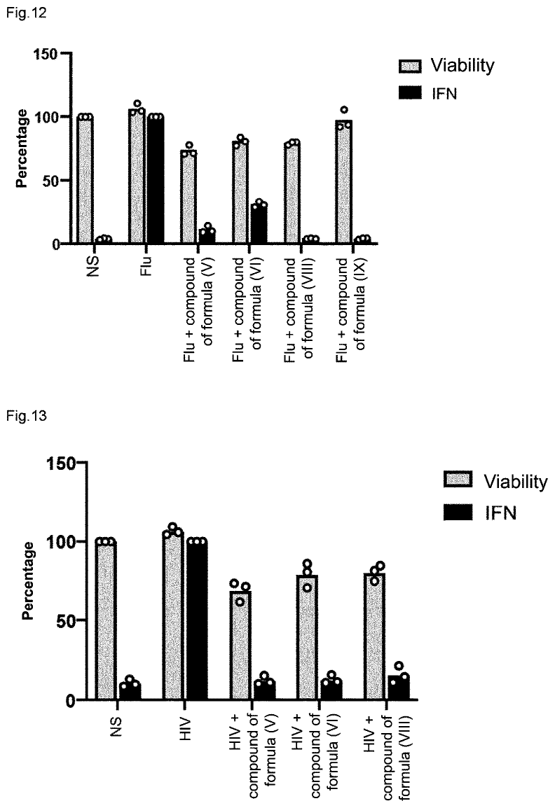

The results are presented in FIG. 12.

### Conclusion

It emerges that the compounds of formula (V), (VI), (VIII) and (IX) inhibit the secretion of interferons by peripheral blood mononuclear cells (PBMCs) from healthy donors, preventively, 1 h before stimulation by the influenza virus.

### Example 5: Effect of the Compounds of Formula (V), (VI) and (VIII) on the Secretion of Interferons by Peripheral Blood Mononuclear Cells (PBMCs) from Healthy Donors, Preventively, 1 h Before Stimulation by the HIV Virus

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). The peripheral blood mononuclear cells (PBMCs) were cultured in RPMI 1640 (Sigma) containing 10% inactivated fetal calf serum and 1 mM glutamine (Hyclone, Logan, UT).

### Cell Stimulation

The PBMCs were cultured at 2×10⁶ cells/ml. The cells were then incubated with the compounds of formula (V), (VI) and (VIII) at 20 μM for 1 hour before stimulation. The PBMCs were then stimulated for 24 h with the human immunodeficiency virus (HIV). The supernatants were collected for cytokine detection.

### Test of Compound Toxicity on the Peripheral Blood Mononuclear Cells (PBMCs)

After 24 h of cell culture, cell viability was determined on the PBMCs using the CellTiter-Glo reagent (Promega), which evaluates the amount of ATP by luminescence using the EnSpire.

### Cytokine Detection

The induction of the interferon (IFN) pathway via the IRFs was measured using the THP1-Dual reporter cell line from InvivoGen. The PBMC culture supernatants were added to THP1-Dual cultures at 0.4×10⁶ cells/ml for 24 h. The activity of the IRFs was measured with QUANTI-Luc, a luciferase detection reagent following the supplier's recommendations.

The results are presented in FIG. 13.

### Conclusion

It emerges that the compounds of formula (V), (VI) and (VIII) inhibit the secretion of interferons by peripheral blood mononuclear cells (PBMCs) from healthy donors, preventively, 1 h before stimulation by the HIV virus.

### Example 6: Effect of the Compounds of Formula (V) and (VI) on the Production of TNFα on Monocytes Isolated from Peripheral Blood Mononuclear Cells (PBMCs) from Healthy Donors

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). Human monocytes were purified by positive selection with human CD14 microbeads (Miltenyi). The monocytes were cultured in RPMI 1640 (Sigma) containing 10% inactivated fetal calf serum and 1 mM glutamine (Hyclone, Logan, UT).

### Cell Stimulation

The monocytes were cultured at 1×10⁶/ml. The cells were then incubated with the compounds of formula (V) and (VI) at 20 μM for 1 hour before stimulation.

The monocytes were stimulated respectively for 16 h with:
condition A: the TLR7/8 agonist Resiquimod—R848 at 5 μg/ml,
condition B: the TLR4 agonist Lipopolysaccharide—LPS at 100 ng/ml,
condition C: the TLR2 agonist lipoteichoic acid—LTA at 1 μg/ml.

The cells were recovered for flow cytometry. For the intracellular labeling of TNFα, Brefeldin A (BFA) was added to the cells 30 minutes after stimulation.

### Flow Cytometry

The cells were rinsed in PBS and then incubated with a viability marker (Zombie Aqua, Biolegend) for 30 min at room temperature. After washing, the cells were resuspended in PBS containing 2% of FCS and 2 mM of EDTA and then labeled with the anti-CD14 antibody (clone REA599) from Miltenyi Biotec, used at 1/100°. For the intracellular labeling of TNFα, the “Inside Stain” kit (Miltenyi Biotec) was used according to the manufacturer's protocol. The cells were fixed for 20 min at room temperature with 250 μL of the Inside Fix solution and then labeled in 100 μL of the Inside Perm solution containing the anti-TNFα antibody (clone cA2, Milteniy Biotec) at 1/50° for 30 min at room temperature. The data acquisition was carried out on the Canto II flow cytometer using Diva software (BD Biosciences, San Jose, CA). FlowJo software was used to analyze the data.

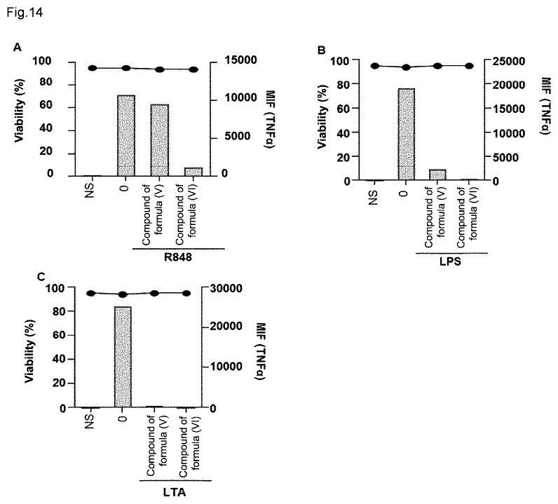

The results are presented in FIG. 14.

### Conclusion

It emerges that the compounds of formula (V) and (VI) inhibit the production of TNFα on monocytes isolated from peripheral blood mononuclear cells (PBMCs) from healthy donors, preventively under the three conditions tested, namely:
Condition A: 1 h before stimulation of TLR7/8,
Condition B: 1 h before stimulation of TLR4,
Condition C: 1 h before stimulation of TLR2.

### Example 7: Effect of the Compounds of Formula (V) and (VI) on the Production of TNFα on Plasmacytoid Dendritic Cells Isolated from Peripheral Blood Mononuclear Cells (PBMC) from Healthy Donors

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Studies on patient material were approved by the personal data protection committee (ID-RCB/EUDRACT: 2014-A01017-40 and 2018-A01358-47). The plasmacytoid dendritic cells (pDCs) were purified by negative selection with the “EasySep Human Plasmacytoid DC” enrichment Kit (STEMCELL Technologies). The plasmacytoid dendritic cells (pDCs) were cultured in RPMI 1640 (Sigma) containing 10% inactivated fetal calf serum and 1 mM glutamine (Hyclone, Logan, UT).

### Cell Stimulation

The plasmacytoid dendritic cells (pDCs) were cultured at 1×10⁶/ml. The cells were then incubated with the compounds of formula (V) and (VI) at 20 μM for 1 hour before stimulation.

The plasmacytoid dendritic cells were stimulated for 16 h, respectively, with:
condition A: the TLR7/8 agonist Resiquimod—R848 at 5 μg/ml,
condition B: the influenza virus (flu),
condition C: the TLR9 agonist CpG-A at 5 mM.

The cells were recovered for flow cytometry. For the intracellular labeling of TNFα, Brefeldin A (BFA) was added to the cells 30 minutes after stimulation.

### Flow Cytometry

The cells were rinsed in PBS and then incubated with a viability marker (Zombie Aqua, Biolegend) for 30 min at room temperature. After washing, the cells were resuspended in PBS containing 2% of FCS and 2 mM of EDTA and then labeled with the anti-CD14 antibody (clone REA599) from Miltenyi Biotec, used at 1/100°. For the intracellular labeling of TNFα, the “Inside Stain” kit (Miltenyi Biotec) was used according to the manufacturer's protocol. The cells were fixed for 20 min at room temperature with 250 μL of the Inside Fix solution and then labeled in 100 μL of the Inside Perm solution containing the anti-TNFα antibody (clone cA2, Milteniy Biotec) at 1/50° for 30 min at room temperature. The data acquisition was carried out on the Canto II flow cytometer using Diva software (BD Biosciences, San Jose, CA). FlowJo software was used to analyze the data.

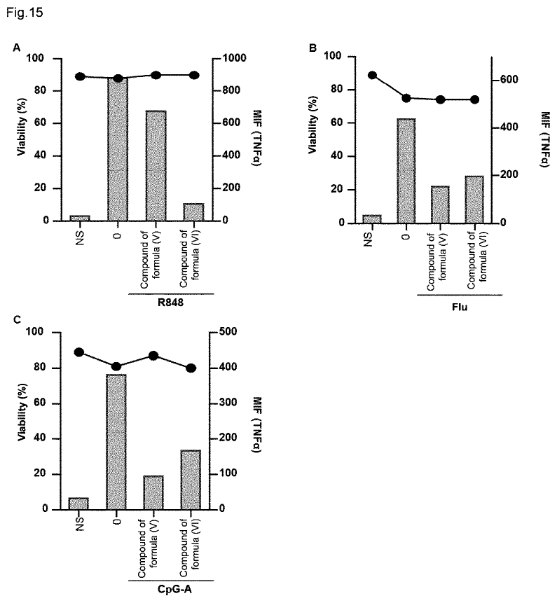

The results are presented in FIG. 15.

### Conclusion

It emerges that the compounds of formula (V) and (VI) inhibit the production of TNFα on plasmacytoid dendritic cells isolated from peripheral blood mononuclear cells (PBMCs) from healthy donors, preventively under the three conditions tested, namely:
Condition A: 1 h before stimulation of TLR7,
Condition B: 1 h before stimulation by the influenza virus (flu virus),
Condition C: 1 h before stimulation of TLR9.

### Example 8: Effect of the Compounds of Formula (V) and (VI) According to a Dose Effect, of the Activation of the NF-κB Transcription Factor in the THP1-Dual Monocytic Cell Line

The THP1-Dual reporter cell line was cultured at 5×10⁵/ml.

The cells were then incubated either with the compound of formula (V), or with the compound of formula (VI) at the indicated concentrations for 1 hour before stimulation.

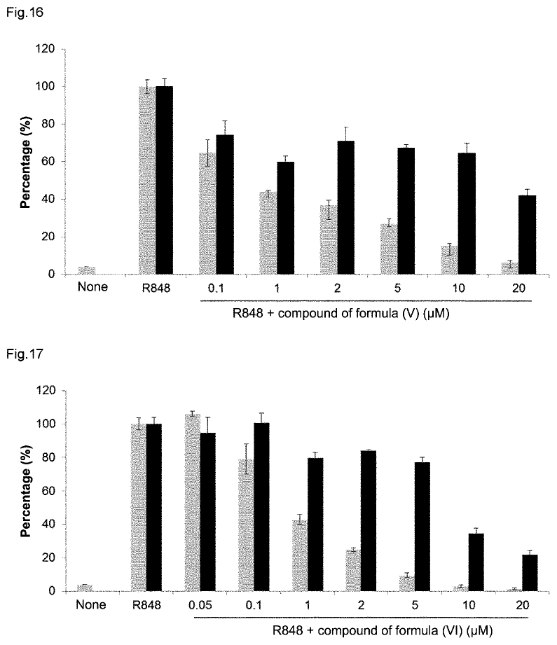

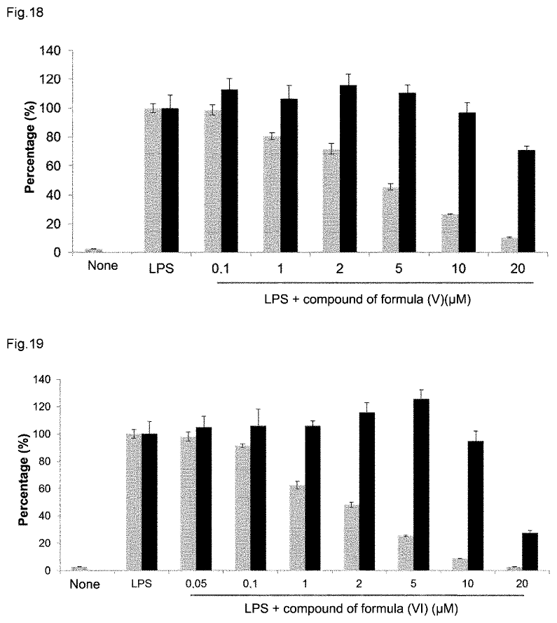

The THP1-Dual cells were stimulated for 24 h with:
the TLR7/8 agonist Resiquimod (R848) at 5 ng·mL (see FIGS. 16 and 17),
the TLR4 agonist Lipopolysaccharide (LPS) at 100 ng·mL (see FIGS. 18 and 19)

### SEAP Assay

The THP1-Dual cells express the secreted embryonic alkaline phosphatase (SEAP) reporter gene under a promoter containing five copies of the NF-κB consensus transcriptional response element. The THP1-Dual cells allow the NF-κB pathway to be studied by monitoring the SEAP activity. The SEAP alkaline phosphatase activity is determined by means of the QUANTI-Blue colorimetric enzyme assay (InvivoGen). The THP1-Dual cells are cultured in a 96-well plate (200 μL/well). After LPS or R848 treatment, for each well, 20 μL of culture medium are added to 180 μL of the QUANTI-Blue solution and incubated at 37° C. for 2 h. Absorbance (measurement at 655 nm with a spectrophotometer (Bio-Rad Model 680 microplate reader) is proportional to SEAP activity.

The results are presented in FIGS. 16 to 19.

### WST Assay

The cells are cultured in a 96-well plate (200 μL/well). After LPS or R848 treatment, for each well, 80 or 90 μL of culture medium are removed and 10 μL of a solution of WST-1 tetrazolium salt (4-(3-(4-iodophenyl)-2-(4-nitrophenyl)-2H-5-tetrazolo)-1,3-benzene disulfonate) (Merck) are added to the remaining 100 μL of medium. The WST-1 is cleaved by the reductases of the respiratory chain of the mitochondria of living cells to give violet-colored and water-soluble formazan which acts as an indicator of cell viability. After 2 h of incubation at 37° C., the absorbance (measurement at 450 and 655 nm with a spectrophotometer (Bio-Rad Model 680 microplate reader) is proportional to the number of viable cells.

The results are presented in FIGS. 16 to 19.

### Conclusion

It emerges that the compounds of formula (V) and (VI) inhibit the activation of the NF-κB transcription factor in the THP1-Dual monocytic cell line, according to a dose effect, in response either to the stimulation of toll-like receptors TLR7/8 by R848, or to the stimulation of toll-like receptors TLR4 by LPS.

### Example 9: Effect of the Compounds of Formula (V), (VI), (VIII) and (IX) According to a Dose Effect, of the Activation of the IRF Transcription Factor in the THP1-Dual Monocytic Cell Line

### Cell Stimulation

The THP1-Dual reporter cell line was cultured at 5×10⁵/ml. The cells were then incubated with the compounds of formula (V), (VI), (VIII) and (IX) at the indicated concentrations for 1 hour before stimulation. The THP1-Dual cells were stimulated for 24 h with the STING pathway agonist, cGAMP. Luciferase assay (luminometry)

The THP1-Dual cells express the Lucia luciferase reporter gene under a promoter containing five IFN response elements. The THP1-Dual cells allow the IRF pathway to be studied by measuring Lucia luciferase activity. Lucia luciferase activity is determined by means of the QUANTI-Luc Gold assay (InvivoGen). The THP1-Dual cells are cultured in a 96-well plate (200 μL/well). After LPS or R848 treatment, for each well, 10 μL of culture medium are added to 50 μL of QUANTI-Luc GOLD solution and the light signal produced is measured by means of a luminometer.

### WST Assay

The cells are cultured in a 96-well plate (200 μL/well). After LPS or R848 treatment, for each well, 80 or 90 μL of culture medium are removed and 10 μL of a solution of WST-1 tetrazolium salt (4-(3-(4-iodophenyl)-2-(4-nitrophenyl)-2H-5-tetrazolo)-1,3-benzene disulfonate) (Merck) are added to the remaining 100 μL of medium. The WST-1 is cleaved by the reductases of the respiratory chain of the mitochondria of living cells to give violet-colored and water-soluble formazan which acts as an indicator of cell viability. After 2 h of incubation at 37° C., the absorbance (measurement at 450 and 655 nm with a spectrophotometer (Bio-Rad Model 680 microplate reader) is proportional to the number of viable cells.

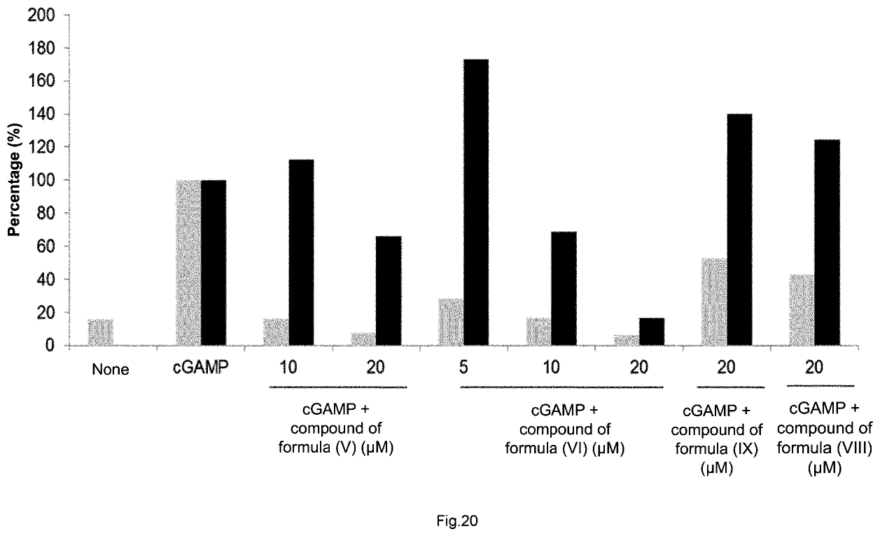

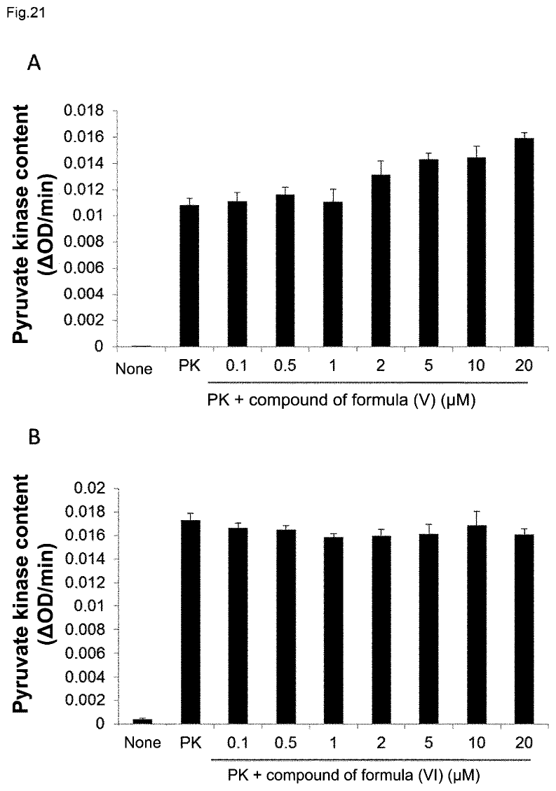

The results are presented in FIG. 20.

### Conclusion

It emerges that the compounds of formula (V), (VI), (VIII) and (IX) inhibit the activation of the IRF transcription factor in the THP1-Dual monocytic cell line, according to a dose effect, in response to the stimulation of the STING interferon pathway by the cGAMP.

### Example 10: Pyruvate Kinase Activity Assay

$$PEP + ADP {⟶}^{Pyruvate kinase} Pyruvate + ATP \\ Pyruvate + β - NADH {⟶}^{L - lactic dehydrogenase} L - lactate + β - NAD \\ PEP = Phospho ( enol ) pyruvate \\ ADP = Adenosine {5}^{′} - Diphosphate \\ ATP = Adenosine {5}^{′} - Triphosphate \\ β - NADH = β - Nicotinamide Adenine Dinucleotide , \\ Reduced Form β - NAD = β - Nicotinamide Adenine Dinucleotide , \\ Oxidized Form$$

Inhibitors (2 μL) are added to 193 μL of a mixture containing PEP, ADP, MgSO4, β-NADH and L-lactic dehydrogenase. After reading at 340 nm, 7 μL of pyruvate kinase are added to the mixture. The samples are read at 340 nm every 1 to 2 min for 20 min.

The results are presented in FIG. 21.

### Conclusion

It emerges that the compounds of formula (V) and (VI) have no impact on the enzymatic activity of pyruvate kinase (PK).

### Example 11: Specific Anti-Inflammatory Effect of the Compounds of Formula (V) and (VI) on the Immune Cells

### Culture

The THP1-Dual cell lines were cultured at 1×10⁶ cells/ml. The cells were then incubated with the compound of formula (V) or the compound of formula (VI) at the indicated concentrations for 1 hour before stimulation.

### Cell Stimulation

The THP1-Dual cells (InvivoGen) were stimulated for 16 h with the TLR7/8 agonist (R848) at 5 μg/ml or for 1 hour with the TLR4 agonist (LPS) at 100 ng·ml, respectively. The cells were washed in PBS and lysed to prepare the mRNAs for an RT-qPCR analysis.

Detection of the mRNAs Encoding TNFα, IL-6, IL-1β and MCP1 by RT-qPCR

The total RNAs are extracted from the cells with the E.Z.N.A. kit. Total RNA Kit 1 (Omega Bio-tek) according to the supplier's instructions. The RNA concentration is measured by means of a spectrophotometer (Nanodrop ND-1000). The total RNA is reverse-transcribed into cDNA by means of the PrimeScript RT Master Mix kit (Takara RR036A). The cDNA synthesis is carried out according to the following protocol: 300-500 ng of RNA in 8 μL of water free of RNase, supplemented with 2 μL of the reaction buffer (containing the PrimeScript reverse transcriptase, RNase inhibitors, oligo(dT) primers, random 6mers and the mix of dNTPs). The enzymatic reverse transcription reaction is carried out at 37° C. for 15 min. The enzyme is inactivated by incubating the sample at 85° C. for 15 sec.

The quantitative real-time PCR is carried out according to the following conditions: cDNA diluted to 1/10^{th} is added to the following mixture: 3.8 μL of H₂O, 0 μL of sense and antisense primers (10 μM) (Table 3), 5 μL of Absolute qPCR SYBR Green Mix containing Thermo-Start DNA Polymerase, MgCl₂, SYBR Green and dNTPs (Eurogentec). The assays are carried out in triplicate in a 384-well plate (Thermo Scientific). DNA amplification by quantitative PCR is carried out in the CFX384 system (Bio-Rad) with the following program: 3 min at 95° C./5 sec at 95° C. and 30 sec at 60° C. repeated 40 cycles/2 min at 72° C. and 30 sec at 95° C. followed by a dissociation step which makes it possible to verify the specificity of the amplification product. The DNA amplification data are analyzed with Bio-Rad CFX Manager software.

TABLE 3
Table 3: Sequence of the primers used

| Name | Sense primer | Antisense primer |
|---|---|---|
| Homo_ | AACAGCTCATGAGGCTAC | ITGGGTCTTGAGGACCTCTGT |
| RPL13A | GG | (SEQ ID No. 2) |
| | (SEQ ID No. 1) | |
| Homo_ | CCTGTCCTGCGTGTTGAA | GGGAACTGGGCAGACTCAAA |
| IL-1β | AGA | (SEQ ID No. 4) |
| | (SEQ ID No. 3) | |
| Homo_ | CCTGCTGCACTTTGGAGT | GAGGGTTTGCTACAACATGGG |
| TNFα | GA (SEQ ID No. 5 | (SEQ ID No. 6) |
| Homo_ | GACAGCCACTCACCTCTT | CCTCTTTGCTGCTTTCACAC |
| IL-6 | CA (SEQ ID No. 7) | (SEQ ID No. 8) |
| Homo_ | AGAATCACCAGCAGCAAG | ATGGAATCCTGAACCCACTTC |
| MCP1 | TGTCC (SEQ ID No. 9) | TGC (SEQ ID No. 10) |

### Results:

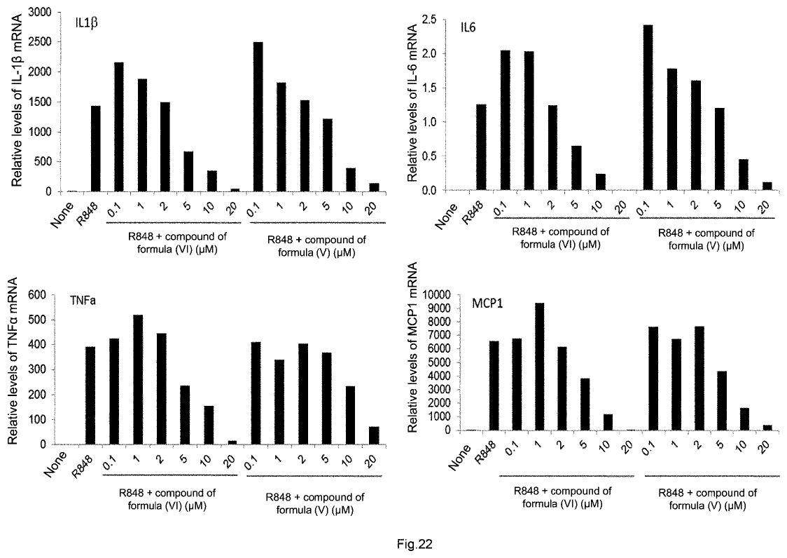

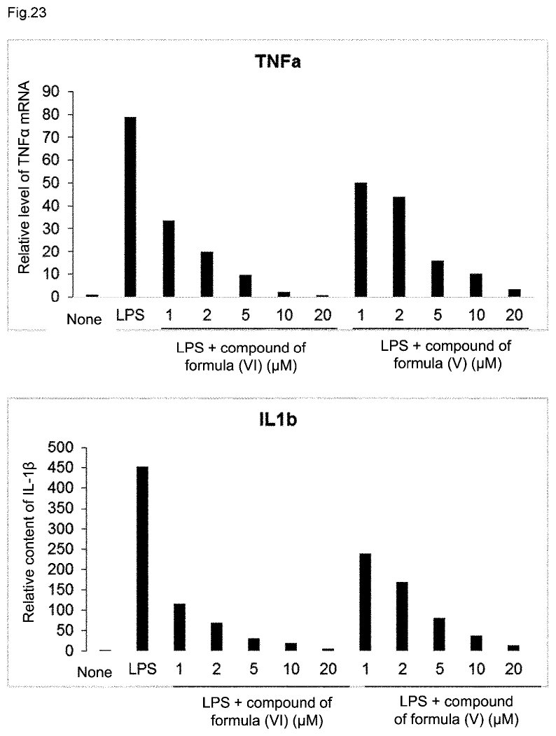

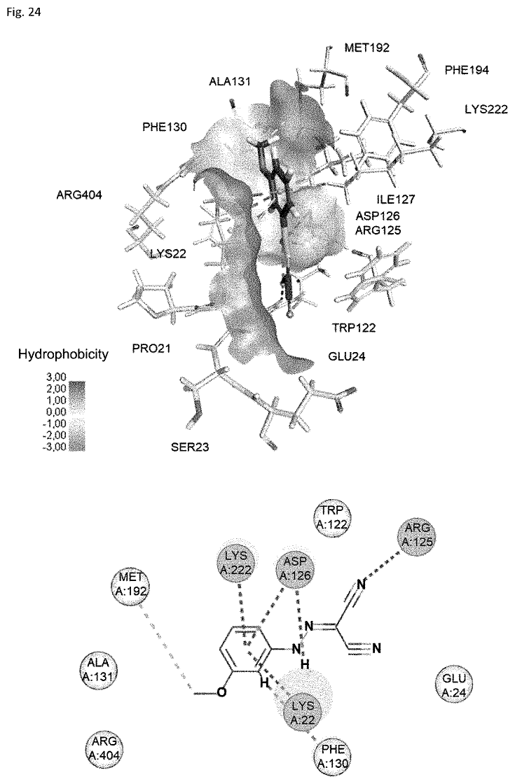

The results are presented in FIGS. 22 and 23.

The anti-inflammatory activity of the compounds of formula (V) and (VI) was tested on the THP1 human monocytic cell line (FIGS. 22 and 23). These cells were stimulated by a TLR7/8 agonist (R848), an agent mimicking an infection with a single-stranded RNA virus.

The compound of formula (V) reduces the transcription of the gene coding for TNFα, IL-6, MCP1 and IL-1β induced by R848, according to a dose effect, from 5 μM for TNFα, IL-6, MCP1 and IL-1β in the THP1-Dual monocytes. The compound of formula (VI) reduces the transcription of the gene coding for TNFα, IL-6, MCP1 and IL-1β induced by R848, according to a dose effect, from 2 μM for TNFα, IL-6, MCP1 and IL-1β in the THP1-Dual monocytes.

These results confirm that the compounds of formula (V) and (VI) exert an immunosuppressive effect, which, in a dose-dependent manner, is specific to the immune cells.

The anti-inflammatory activity of the compounds of formula (V) and (VI) was tested on a THP1 human monocytic cell line, activated with LPS, ligand of the toll-like receptors of type 4 (TLR4), an agent which mimics a bacterial infection. The activation of this pathway induces the production of pro-inflammatory cytokines including TNFα, IL-1 as a result of the transcriptional stimulation of the gene coding for TNFα and IL-1β.

The compound of formula (V) reduces the transcription of the gene coding for TNFα and IL-1β induced by LPS, according to a dose effect, from 5 μM for TNFα and IL-1β in the THP1-Dual monocytes. The compound of formula (VI) reduces the transcription of the gene coding for TNFα and IL-1β induced by LPS, according to a dose effect, from 1 μM for TNFα and IL-1β in the THP1-Dual monocytes.

These results confirm that the compounds of formula (V) and (VI) exert an immunosuppressive effect, which, in a dose-dependent manner, is specific to the immune cells.

### Example 12: Validation of the ROCK-PDK1 Target

### Isolation and Culture of Blood Leukocytes

The in vitro experiments were carried out using human peripheral blood mononuclear cells (PBMCs) isolated by density with centrifugation using a medium for separating white blood cells from peripheral blood (STEMCELL Technologies). The blood from the healthy donors was obtained at the “Etablissement Frangais du Sang” (French Blood Establishment) (convention #07/CABANEL/106; Paris, France). Human monocytes were purified by positive selection with human CD14 microbeads (Miltenyi). The monocytes were cultured in RPMI 1640 (Sigma) containing 10% inactivated fetal calf serum and 1 mM glutamine (Hyclone, Logan, UT).

Treatment of Monocytes with siRNAs

The monocytes were cultured at 1×10⁶/ml and then treated with siRNAs at 160 nM, which target the mRNA coding for the PDK1 kinase (siPDK1) (Dharmacon), or with control siRNAs (siCTL) at 160 nM (Qiagen) coupled to a transfection agent for 24 h.

### Cell Stimulation

The cells were incubated with the compounds of formula (V) and (VI) at 20 μM for 1 hour before stimulation with the TLR7/8 agonist R848 at 1 μg/ml. The monocytes were analyzed by flow cytometry. For the intracellular labeling of TNFα, Brefeldin A (BFA) was added to the cells 30 minutes after R848 stimulation.

### Flow Cytometry

The cells were rinsed in PBS and then incubated with a viability marker (Zombie Aqua, Biolegend) for 30 min at room temperature. After washing, the cells were resuspended in PBS containing 2% of FCS and 2 mM of EDTA and then labeled with the anti-CD14 antibody (clone REA599) from Miltenyi Biotec, used at 1/100°. For the intracellular labeling of TNFα, the “Inside Stain” kit (Miltenyi Biotec) was used according to the manufacturer's protocol. The cells were fixed for 20 min at room temperature with 250 μL of the Inside Fix solution and then labeled in 100 μL of the Inside Perm solution containing the anti-TNFα antibody (clone cA2, Milteniy Bioec) at 1/50° for 30 min at room temperature. The data acquisition was carried out on the Canto II flow cytometer using Diva software (BD Biosciences, San Jose, CA). FlowJo software was used to analyze the data.

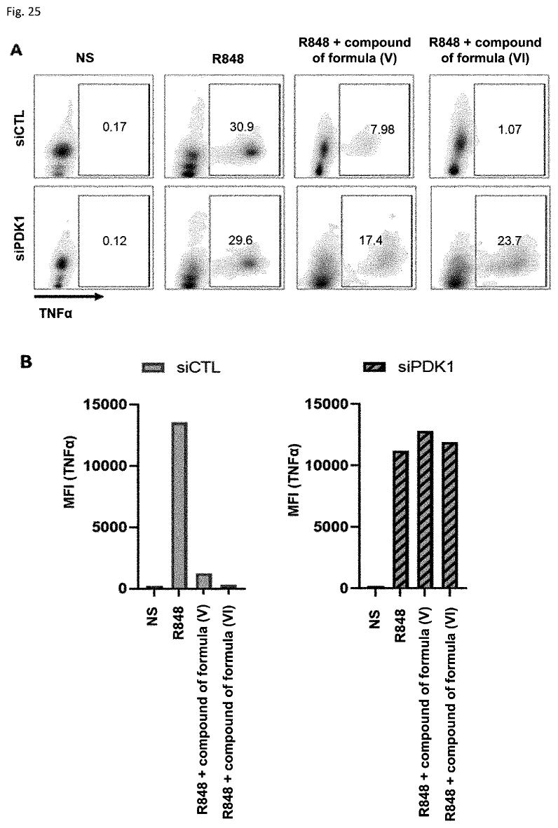

The results are presented in FIG. 25.

### CONCLUSION

It emerges that the compounds of formula (V) and (VI) inhibit the production of TNFα by the monocytes from healthy donors, treated with the control siRNAs (siCTL) and stimulated with R848 (TLR7/8 agonist). Conversely, in the monocytes treated with siRNAs targeting the PDK1 kinase (siPDK1), the compounds of formula (V) and (VI) fail to inhibit the production of TNFα induced by R848 (see FIG. 25). These results demonstrate that the PDK1 kinase relays the anti-inflammatory action of the compounds of formulas (V) and (VI) directed against the ROCK kinase, validating the ROCK-PDK1 complex as a target provided with anti-inflammatory activity.

1. A method for prevention and/or treatment of a disease requiring the use of an inhibitor of the interaction between the ROCK protein kinase and the PDK1 protein kinase in a human, comprising administering a therapeutically effective amount of a compound of formula (I) to the human: 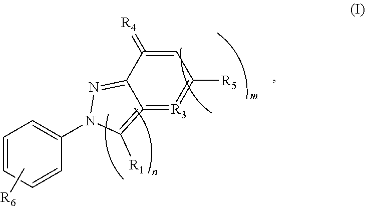
or a pharmaceutically acceptable salt thereof,
m is selected from 0, 1 and 2,
n is selected from 0, 1 and 2,
R₁ is a —COR₂ group, wherein R₂ is selected from the group consisting of an —OH group, an —NH₂ group, an alkoxy group, an alkylamine group, an arylalkylamine group, an —NH—NH₂ group, and a —CH₂—Ar group substituted or non-substituted by at least one halogen atom, wherein Ar is an aryl group,
R₃ is a nitrogen atom, an —NHCOR₇ group, an —NHCOORₛ group, or a —COSR₉ group, wherein R₇, Rₛ and R₉ are C₁ to C₆ alkyl groups substituted or non-substituted by at least one halogen atom,
R₄ is a nitrogen atom or an —NH₂ group,
R₅ and R₆, which are identical or different, are selected independently from each other in the group consisting of a hydrogen atom, an —OH group, an —NH₂ group, an alkoxy group, a thiol group, a sulfonamide group, an alkylamine group, and an aryl group substituted or non-substituted by at least one halogen atom, and wherein:
the lines [symbol] and [symbol] , which are identical or different, depict independently of each other, a single covalent bond or a triple covalent bond.

2. The method of claim 1, wherein R₆ can be in position 3, 4 or 5 on the ring.

3. The method of claim 1, wherein m is 0 and n is 0, and wherein the bonds [symbol] and [symbol] depict a triple covalent bond.

4. The method of claim 1, wherein the compound of formula (I) is a compound of formula (II) or a pharmaceutically acceptable salt thereof, 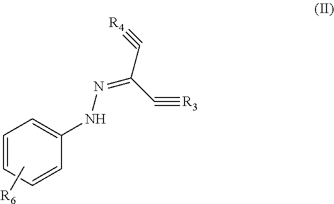
wherein R3, R4 and R6 are as defined in formula (I) of claim 1.

5. The method of claim 1, wherein m is 1 and n is 1, and wherein the bonds [symbol] and [symbol] depict a single covalent bond.

6. The method of claim 1, wherein the compound of formula (I) is a compound of formula (III) or a pharmaceutically acceptable salt thereof, 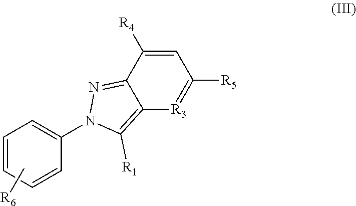
wherein R1, R3, R4, R5 and R6 are as defined in formula (I) of claim 1.

7. The method of claim 1, wherein m is 0 and n is 1, and wherein the bond [symbol] depicts a triple covalent bond and the bond [symbol] depicts a single covalent bond.

8. The method of claim 1, wherein the compound of formula (I) is a compound of formula (IV) or a pharmaceutically acceptable salt thereof, 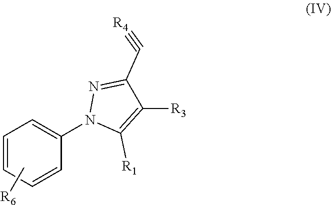
wherein R1, R3, R4 and R6 are as defined in formula (I) of claim 1.

9. The method of claim 1, wherein the compound of formula (I) is

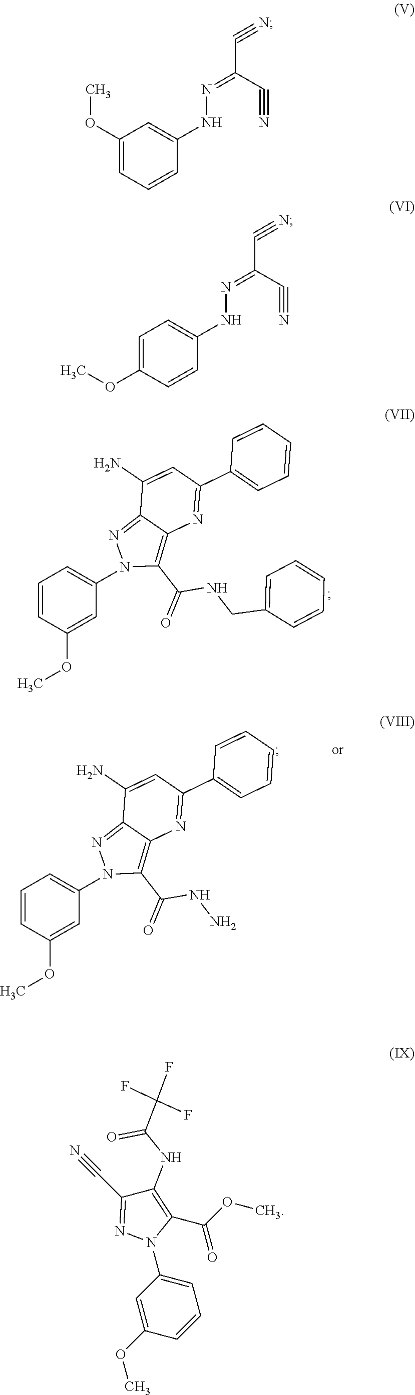 [SMILES: COc1cccc(NN=C(C#N)C#N)c1 ; COc1ccc(NN=C(C#N)C#N)cc1 ; COc1cccc(-n2nc3c(N)cc(-c4ccccc4)nc3c2C(=O)NCc2ccccc2)c1 ; COc1cccc(-n2nc3c(N)cc(-c4ccccc4)nc3c2C(=O)NN)c1 ; COC(=O)c1c(NC(=O)C(F)(F)F)c(C#N)nn1-c1cccc(OC)c1]

10. A pharmaceutical composition comprising at least one compound according to claim 1 as an active ingredient and at least one pharmaceutically acceptable excipient and/or a pharmaceutically acceptable support and/or a diluent and/or a carrier.

11. The pharmaceutical composition according to claim 10, wherein the pharmaceutical composition further comprises at least a second active ingredient.

12. A method for prevention and/or treatment of a disease requiring the use of an inhibitor of the ROCK enzyme and/or of the PDK1 enzyme in a human, comprising administering the pharmaceutical composition of claim 10 to the human.

13. The method of claim 1, wherein the disease requiring the use of an inhibitor of the ROCK enzyme and/or of the PDK1 enzyme is selected from: inflammatory diseases, viral infections, bacterial infections and/or autoimmune diseases.

14. The method of claim 12, wherein the disease requiring the use of an inhibitor of the ROCK enzyme and/or of the PDK1 enzyme is selected from: inflammatory diseases, viral infections, bacterial infections and/or autoimmune diseases.


SEQUENCE LISTING (11 sequences)

SEQ ID NO: 1 - DNA, 20 bp, synthetic construct
  source 1..20: mol_type = other DNA
  misc_feature 1..20: note = Amorce sens
aacagctcatgaggctacgg

SEQ ID NO: 2 - DNA, 20 bp, synthetic construct
  source 1..20: mol_type = other DNA
  misc_feature 1..20: note = Amorce anti-sens
tgggtcttgaggacctctgt

SEQ ID NO: 3 - DNA, 21 bp, synthetic construct
  source 1..21: mol_type = other DNA
  misc_feature 1..21: note = Amorce sens
cctgtcctgcgtgttgaaaga

SEQ ID NO: 4 - DNA, 20 bp, synthetic construct
  source 1..20: mol_type = other DNA
  misc_feature 1..20: note = Amorce anti-sens
gggaactgggcagactcaaa

SEQ ID NO: 5 - DNA, 20 bp, synthetic construct
  source 1..20: mol_type = other DNA
  misc_feature 1..20: note = amorce sens
cctgctgcactttggagtga

SEQ ID NO: 6 - DNA, 21 bp, synthetic construct
  source 1..21: mol_type = other DNA
  misc_feature 1..21: note = Amorce anti-sens
gagggtttgctacaacatggg

SEQ ID NO: 7 - DNA, 20 bp, synthetic construct
  source 1..20: mol_type = other DNA
  misc_feature 1..20: note = Amorce sens
gacagccactcacctcttca

SEQ ID NO: 8 - DNA, 20 bp, synthetic construct
  source 1..20: mol_type = other DNA
  misc_feature 1..20: note = Amorce anti-sens
cctctttgctgctttcacac

SEQ ID NO: 9 - DNA, 23 bp, synthetic construct
  source 1..23: mol_type = other DNA
  misc_feature 1..23: note = Amorce sens
agaatcaccagcagcaagtgtcc

SEQ ID NO: 10 - DNA, 24 bp, synthetic construct
  source 1..24: mol_type = other DNA
  misc_feature 1..24: note = Amorce anti-sens
atggaatcctgaacccacttctgc

SEQ ID NO: 11 - protein, 415 aa, Homo sapiens
GSLHMSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNR